(19) United States
(12) Patent Application Publication
(10) Pub. No.: US 2019/0023783 A1
(43) Pub. Date: Jan. 24, 2019

(54) METHOD OF TREATING CANCER

(71) Applicants: CSL Limited, Parkville (AU); Ludwig Institute for Cancer Research Ltd, New York, NY (US)

(72) Inventors: Andreas BEHREN, New York, NY (US); Jonathan CEBON, New York, NY (US); Christopher HUDSON, New York, NY (US); Katherine WOODS, New York, NY (US); Andrew HAMMET, Parkville (AU); Anne VERHAGEN, Parkville (AU); Eugene MARASKOVSKY, Parkville (AU); Con PANOUSIS, Parkville (AU)

(21) Appl. No.: 16/051,250

(22) Filed: Jul. 31, 2018

Related U.S. Application Data
(63) Continuation of application No. 15/039,839, filed on May 26, 2016, now abandoned.

(30) Foreign Application Priority Data
Nov. 29, 2013 (AU) 2013904620

(51) Int. Cl. C07K 16/28; C07K 16/30; A61K 47/68; C12N 15/113

(52) U.S. Cl. CPC C07K 16/2803; C07K 16/3053; A61K 47/6851; A61K 47/6849; C07K 2317/76; C07K 2319/00; C12N 15/1138; C07K 2317/21; C07K 2317/24; C07K 2317/52; C07K 2317/54; C07K 2317/55; C07K 2317/56; C07K 2317/569; C07K 2317/622; C07K 2317/626; C07K 2317/734; C12N 2310/14; C07K 2317/92; C07K 2317/732; C07K 16/30

(57) ABSTRACT

The present disclosure provides computer-implemented methods of organising the marketing of a compound that neutralises BTN2A1.

### RELATED APPLICATION

The present application claims priority from Australian Patent Application No. 2013904620, filed on 29 Nov. 2013 and entitled “Method of treating cancer”. The entire contents of that earlier application are hereby incorporated by reference.

### FIELD

The present disclosure relates to reagents and methods for treating cancer.

### INTRODUCTION

In spite of numerous advances in medical research, cancer remains the second leading cause of death in the United States. Traditional modes of clinical care, such as surgical resection, radiotherapy and chemotherapy, have a significant failure rate, especially for solid tumors. Failure occurs either because the initial tumor is progressed too far for complete surgical removal, is unresponsive, or because of recurrence due to regrowth at the original site or metastasis. Cancer remains a central focus for medical research and development.

Three major cancers, in terms of morbidity and mortality, are colon cancer, prostate cancer and lung cancer. New surgical procedures offer an increased survival rate for colon cancer. Improved screening methods increase the detection of prostate cancer, allowing earlier, less aggressive therapy. Numerous studies have shown that early detection increases survival and treatment options. Lung cancer remains largely refractory to treatment.

Excluding basal cell carcinoma, there are over one million new cases of cancer per year in the United States alone, and cancer accounts for over one half million deaths per year in this country. In the world as a whole, the total number of new cases of cancer per year is over 6 million.

Skin cancer is the most common of all cancers and melanoma is the most serious and aggressive type of skin cancer. Melanoma accounts for less than 5% of skin cancer cases, yet it is responsible for a large majority of the deaths associated with skin cancer. Almost 70,000 people in the United States were diagnosed with melanoma during 2010 and approximately 9,000 people were expected to die from the disease (American Cancer Society; www.cancer.org). Across the world the incidence of melanoma has been increasing, with a lifetime risk of developing melanoma as high as 1/58 for males in the U.S. to 1/25 for males in Australia. Metastatic melanoma remains one of the most difficult cancers to treat and individuals with this advanced form have an average survival time of only nine to eleven months.

It will be clear to the skilled person from the foregoing that new treatments for cancer, e.g., melanoma are desirable.

### SUMMARY

In arriving at the present invention, the inventors identified a membrane-bound protein, butyrophilin, subfamily 2, member A1 (BTN2A1), which is highly expressed on cancer cells, e.g., melanoma cells and at a low level on normal cells. The inventors also produced antibodies against BTN2A1 and showed that antibodies against BTN2A1 were capable of inducing antibody-dependent cell-mediated cytotoxicity (ADCC) thereby killing cells (e.g., melanoma cells). Furthermore, the inventors showed that neutralization of BTN2A1 enhanced immune reaction against melanoma. For example, BTN2A1 was shown to suppress proliferation and activation of CD4⁺ and CD8⁺ T cells, and neutralizing this protein resulted in increased levels of activated T cells and cytotoxicity of melanoma cells. The inventors additionally showed that BTN2A1 protein is expressed on a variety of cancer cells, e.g., colon cancer cells, prostate cancer cells, lung cancer cells and not significantly expressed on normal cells, including normal fibroblasts and blood cells, such as monocytes.

These findings by the inventors provide the basis for reagents that bind to and/or neutralize BTN2A1 and their use in the treatment of cancer, e.g., melanoma, colon cancer, lung cancer or prostate cancer. For example, the present disclosure provides a method comprising administering to the subject a compound that neutralizes BTN2A1 and/or that binds to BTN2A1 on a cell (e.g., a cancer cell, such as a melanoma cell, a colon cancer cell, a lung cancer cell or a prostate cancer cell) and induces death of the cell. For example, the compound is administered to a subject suffering from cancer, e.g., melanoma.

The present disclosure also provides a method for enhancing or inducing an immune response in a subject, the method comprising administering to the subject a compound that neutralizes BTN2A1. In one example, the subject suffers from cancer.

In one example, the subject suffers from melanoma. In one example, the subject suffers from colon cancer. In one example, the subject suffers from lung cancer. In one example, the subject suffers from prostate cancer. Optionally, the compound binds to BTN2A1 on a cell (e.g., a cancer cell, such as a melanoma cell) and induces death of the cell.

The present disclosure also provides a method for inducing lysis of cancer cells, e.g., melanoma cells in a subject, the method comprising administering to the subject a compound that neutralizes BTN2A1. Optionally, the compound binds to BTN2A1 on a cell (e.g., a cancer cell, such as a melanoma cell, a colon cancer cell, a lung cancer cell or a prostate cancer cell) and induces death of the cell.

The present disclosure also provides a method for inducing death of cancer cells, e.g., melanoma cells, colon cancer cells, lung cancer cells or prostate cancer cells in a subject, the method comprising administering to the subject a compound that neutralizes BTN2A1 and/or that binds to BTN2A1 on the cells and induces death of the cells. In one example, the compound is an antibody that induces death by ADCC or by inducing an immune response (e.g., a T cell-mediated immune response) against the cells.

The present disclosure additionally provides a method of treating cancer in a subject, the method comprising administering to the subject a compound that neutralizes BTN2A1 and/or that binds to BTN2A1 on a cell (e.g., a cancer cell) and induces death of the cell.

In one example, the cancer is colon cancer, prostate cancer, lung cancer or melanoma.

In one example, the cancer is melanoma.

In one example, the cancer expresses or overexpresses BTN2A1 (e.g., overexpresses BTN2A1 at the protein level, e.g., on the surface of the cancer cell).

The present disclosure additionally provides a method of treating melanoma in a subject, the method comprising administering to the subject a compound that neutralizes BTN2A1 and/or that binds to BTN2A1 on a cell (e.g., a melanoma cell) and induces death of the cell.

In one example, the melanoma is primary melanoma or unresectable melanoma or metastatic melanoma.

In one example, the melanoma expresses or overexpresses BTN2A1 (e.g., overexpresses BTN2A1 at the protein level, e.g., on the surface of the melanoma cell).

The present disclosure additionally provides a method of treating colon cancer in a subject, the method comprising administering to the subject a compound that neutralizes BTN2A1 and/or that binds to BTN2A1 on a cell (e.g., a colon cancer cell) and induces death of the cell.

In one example, the colon cancer expresses or overexpresses BTN2A1 (e.g., overexpresses BTN2A1 at the protein level, e.g., on the surface of the colon cancer cell).

The present disclosure additionally provides a method of treating lung cancer in a subject, the method comprising administering to the subject a compound that neutralizes BTN2A1 and/or that binds to BTN2A1 on a cell (e.g., a lung cancer cell) and induces death of the cell.

In one example, the lung cancer expresses or overexpresses BTN2A1 (e.g., overexpresses BTN2A1 at the protein level, e.g., on the surface of the lung cancer cell).

The present disclosure additionally provides a method of treating prostate cancer in a subject, the method comprising administering to the subject a compound that neutralizes BTN2A1 and/or that binds to BTN2A1 on a cell (e.g., a prostate cancer cell) and induces death of the cell.

In one example, the prostate cancer expresses or overexpresses BTN2A1 (e.g., overexpresses BTN2A1 at the protein level, e.g., on the surface of the prostate cancer cell).

In one example, the compound is administered in an amount sufficient to induce cytotoxic killing of the melanoma cells by T cells and/or activate T cells (e.g., as determined by the level of IFNγ or TNFα production).

In one example, the compound is a compound that binds to BTN2A1 on a cell and:

(i) modulates BTN2A1 signaling (e.g., induces or enhances or reduces signalling) and/or
(ii) induces death of the cell.

In one example, the compound is a compound that binds to BTN2A1 on a cell and:

(i) neutralizes BTN2A1 signaling and/or
(ii) induces death of the cell,
as described herein.

In one example, the compound is a protein comprising the extracellular domain of BTN2A1, e.g., fused to an antibody constant region, e.g., an IgG Fc region (optionally, including a hinge region).

In one example, the compound inhibits or prevents expression of BTN2A1. For example, the compound is selected from the group an antisense, a siRNA, a RNAi, a shRNA, and a catalytic nucleic acid, e.g., a ribozyme or a DNAzyme.

In one example, the BTN2A1 is mammalian BTN2A1, e.g., human BTN2A1.

In one example, the subject is a mammal, for example a primate, such as a human.

Methods of treatment described herein can additionally comprise administering a further compound to treat the cancer, e.g., melanoma, prostate cancer, colon cancer or lung cancer. For example, the further compound is an immunotherapy or a chemotherapy.

Methods of treatment described herein can additionally comprise performing an additional treatment to treat the cancer, e.g., melanoma, e.g., surgery and/or radiotherapy.

In one example, a method as described herein additionally comprises detecting BTN2A1 on a cell, e.g., cancer cell, e.g., a melanoma cell, a colon cancer cell, a lung cancer cell or a prostate cancer cell from the subject.

The present disclosure additionally provides for use of a compound that neutralizes BTN2A1 and/or that binds to BTN2A1 on a cell (e.g., a cancer cell) and induces death of the cell in the manufacture of a medicament to treat cancer in a subject.

The present disclosure additionally provides for use of a compound that neutralizes BTN2A1 and/or that binds to BTN2A1 on a cell (e.g., a melanoma cell) and induces death of the cell in the manufacture of a medicament to treat melanoma and/or to enhance or induce an immune response in a subject.

The present disclosure additionally provides a compound that neutralizes BTN2A1 and/or that binds to BTN2A1 on a cell (e.g., a cancer cell) and induces death of the cell for use in treating cancer in a subject.

The present disclosure additionally provides a compound that neutralizes BTN2A1 and/or that binds to BTN2A1 on a cell (e.g., a melanoma cell) and induces death of the cell for use in treating melanoma and/or to induce an immune response in a subject.

The present disclosure additionally provides a compound that neutralizes BTN2A1 and/or that binds to BTN2A1 on a cell (e.g., a colon cancer cell) and induces death of the cell for use in treating colon cancer and/or to induce an immune response in a subject.

The present disclosure additionally provides a compound that neutralizes BTN2A1 and/or that binds to BTN2A1 on a cell (e.g., a prostate cancer cell) and induces death of the cell for use in treating prostate cancer and/or to induce an immune response in a subject.

The present disclosure additionally provides a compound that neutralizes BTN2A1 and/or that binds to BTN2A1 on a cell (e.g., a lung cancer cell) and induces death of the cell for use in treating lung cancer and/or to induce an immune response in a subject.

The present disclosure additionally provides a compound that binds to BTN2A1 on a cell and:

(i) neutralizes BTN2A1 signaling and/or
(ii) induces death of the cell.

In one example, the present disclosure provides a protein comprising an antigen binding domain, wherein the antigen binding domain binds to BTN2A1 on a cell and:

(i) neutralizes BTN2A1 signaling and/or
(ii) induces death of the cell.

In one example, the cell is a melanoma cell.

In one example, the antigen binding domain is an antigen binding domain of an immunoglobulin, e.g., of an antibody.

In one example, the neutralization of BTN2A1 is determined by contacting cancer cells, e.g., melanoma cells with the compound such that the compound binds to the BTN2A1 forming a cell-compound complex; contacting the complex with a T cell (e.g., a CD4⁺ T cell or a CD8⁺ T cell); and determining the level of death of the melanoma cells (e.g., cytotoxic killing of the cancer cells by the T cells), wherein an increase in the level of death of the melanoma cells in the presence of the compound compared to in the absence of the compound indicates that the compound neutralized BTN2A1.

In one example, the neutralization of BTN2A1 is determined by contacting a cancer cell (e.g., melanoma cell) with the compound such that the compound binds to the BTN2A1 forming a cell-compound complex; contacting the complex with T cells (e.g., CD4⁺ T cells or CD8⁺ T cells); and determining the level of activation of the T cells (e.g., by determining the level of intracellular interferon (IFN) γ or tumor necrosis factor (TNF) α), wherein an increase in the level of activation of the T cells in the presence of the compound compared to in the absence of the compound indicates that the compound neutralized BTN2A1.

Compounds contemplated by the present disclosure can take any of a variety of forms including natural compounds, chemical small molecule compounds or biological compounds. Exemplary compounds include a nucleic acid (e.g., an aptamer), a polypeptide, a peptide, a small molecule, an antibody or an antigen binding fragment of an antibody.

In one example, the compound is a protein-based compound, e.g., a peptide, polypeptide or protein.

In one example, the compound is an antibody mimetic. For example, the compound is a protein comprising an antigen binding domain of an immunoglobulin, e.g., an IgNAR, a camelid antibody or a T cell receptor.

In another example, the antibody mimetic is a protein comprising a non-antibody antigen binding domain, such as an adnectin, an affibody, an atrimer, an evasin, a designed ankyrin-repeat protein (DARPin) or an anticalin.

In one example, a compound of the present disclosure is an antibody or an antigen binding fragment thereof. In one example, an antibody of the present disclosure is a monoclonal antibody, a chimeric antibody, a humanized antibody or a human antibody.

In one example, an antibody or antigen binding fragment of the present disclosure is a human antibody or antigen binding fragment thereof.

Exemplary antigen binding fragments contemplated by the present disclosure include:

(i) a domain antibody (dAb);

### (ii) a Fv;

(iii) a scFv or stabilized form thereof (e.g., a disulfide stabilized scFv);
(iv) a dimeric scFv or stabilized form thereof;
(v) a diabody, triabody, tetrabody or higher order multimer;
(vi) Fab fragment;
(vii) a Fab′ fragment;
(viii) a F(ab′) fragment;
(ix) a F(ab′)₂ fragment;
(x) any one of (i)-(ix) fused to a Fc region of an antibody;
(xi) any one of (i)-(ix) fused to an antibody or antigen binding fragment thereof that binds to an immune effector cell (e.g., a bispecific T cell effector/engager; BiTe).

In one example, a compound (e.g., an antibody or antigen binding fragment thereof) of the present disclosure induces death of a cell to which it binds, e.g., cancer cells, such as melanoma cells.

In some example, the compounds (e.g., antibodies) are capable of induce death of cells to which it binds without being conjugated to a toxic compound.

In one example, a compound (e.g., an antibody or antigen binding fragment thereof) of the present disclosure is capable of inducing an effector function, e.g., an effector function that results in death a cell to which the antibody or antigen binding fragment thereof binds. Exemplary effector functions include ADCC, antibody-dependent cell-mediated phagocytosis (ADCP) and/or complement-dependent cytotoxicity (CDC).

In one example, the compound (e.g., the antibody or antigen binding fragment thereof) is capable of inducing ADCC.

In one example, the compound is capable of inducing an enhanced level of effector function. For example, the compound (e.g., the antibody or antigen binding fragment) comprises a Fc region that is afucosylated.

In one example, the compound (e.g., antibody or antigen binding fragment thereof) comprises an Fc region comprising one or more amino acid sequence substitutions that enhance the effector function induced by the compound (e.g., antibody or antigen binding fragment). For example, the one or more amino acid sequence substitutions increase the affinity of the Fc region for a Fcγ receptor (FcγR) compared to a Fc region not comprising the substitutions. For example, the one or more amino acid substitutions enhance increase the affinity of the Fc region for a FcγR selected from the group consisting of FcγRI, FcγRIIa, FcγRIIc and FcγRIIIa compared to a Fc region not comprising the substitutions.

In one example, the compound (e.g., antibody or antigen binding fragment thereof) is conjugated to an agent. Exemplary agents include a detectable label or a compound that extends the half-life of the protein or antibody, such as polyethylene glycol or an albumin binding protein or an agent that induces death of a cell to which the compound binds. Exemplary agents are described herein.

In one example, an antibody of the present disclosure is a full length antibody.

The present disclosure also provides a composition comprising a compound (e.g., an antibody or antigen binding fragment thereof) according to the present disclosure and a pharmaceutically acceptable carrier.

As discussed above, the present inventors have also shown that neutralizing BTN2A1 induces an immune response (e.g., a T cell immune response) that is effective in killing cancer cells, e.g., melanoma cells. Thus, the present inventors have demonstrated a therapeutic effect of compounds that neutralize BTN2A1 (e.g., antagonists of BTN2A1 expression and/or activity) and/or that bind to BTN2A1 on a cell and induce death of the cell. In accordance with this finding, the present disclosure provides a method of treating a disease or disorder comprising administering to a subject suffering from the disease or disorder a compound that neutralizes BTN2A1 and/or that binds to BTN2A1 on a cell and induce death of the cell. Similarly, the present disclosure provides for the use of a compound that neutralizes BTN2A1 and/or that binds to BTN2A1 on a cell and induces death of the cell in the manufacture of a medicament or in medicine.

### KEY TO SEQUENCE LISTING

SEQ ID NO: 1 is an amino acid sequence of human BTN2A1 isoform 1.
SEQ ID NO: 2 is an amino acid sequence of human BTN2A1 isoform 2.
SEQ ID NO: 3 is an amino acid sequence of human BTN2A1 isoform 3.
SEQ ID NO: 4 is an amino acid sequence of human BTN2A1 isoform 4.

### BRIEF DESCRIPTION OF DRAWINGS

FIG. 1 is a graphical representation showing expression of BTN2A1, PD1L1 and PD1L2 in melanoma tumors. Data are expressed as absolute counts and the solid line represents cut-off of 50 counts as usually used for analysis.

FIGS. 2A-G are graphical representations showing results of flow cytometry analysis (FIGS. 2A-E) or microarray analysis (FIGS. 2F and G) of BTN2A1 expression on melanoma cell lines (FIG. 2A), colon cancer cell lines (FIG. 2B), lung cancer cell lines (FIG. 2C), prostate cancer cell lines (FIG. 2D), monocytes (FIG. 2E), normal tissues (FIG. 2F) and melanoma cells (FIG. 2G). For FIGS. 2A-E, the names of the cell lines are included in the tables to the right of each graph and results with anti-BTN2A1 antibody are shown in light grey and results with an isotype control antibody are shown in dark grey.

FIG. 3 includes a series of graphical representations labelled A-C, and shows that BTN2A1 inhibits T cell proliferation and induces FoxP3⁺ regulatory T cells. In FIGS. 3A and 3B, 96 well plates were coated with recombinant BTN2A1 (10 μg/ml) or BSA (10 μg/ml) and PBMCs (CSFE-labelled) added. After 5 days the percentage of proliferating CD4⁺ (FIG. 3A) or CD8⁺ (FIG. 3B) cells was analysed by flow cytometry. In FIG. 1C, PBMCs or sorted CD4⁺ cells were added into BTN2A1 or BSA coated plates and incubated for 3 days. The percentage of CD4/CD25/FoxP3⁺ cells was measured by flow cytometry.

FIG. 4 includes a series of graphical representations labeled A-D, and shows BTN2A1 knockdown in tumor cells leads to an increase in T cell activation and a higher tumor cell clearance. In FIG. 2A melanoma cells were transfected with siRNA specific for BTN2A1 or a scrambled siRNA control and BTN2A1 expression measured by flow-cytometry after 48h. In FIG. 2B two days after BTN2A1 knockdown in NY-ESO-1 positive melanoma cells, HLA-matched T cells recognizing epitope 96-104 were added to the culture and surviving melanoma cells measured by MTS after 20 h. FIGS. 2C and 2D show levels of intracellular cytokines (IFNγ (FIG. 2C) or TNFα (FIG. 2D)) in CD8⁺ T cells after co-incubation with melanoma cells in the same conditions as described for (B). Levels observed for scrambled control was set to 1 on the Y axis.

FIG. 5 includes two graphical representations labeled A and B, and shows results of an in vitro ADCC assay. BTN2A1-positive target cells [either LM-MEL62 (A) or 293FS (B)] were incubated with anti-BTN2A1 antibody (34C1) and NK cells at an effector to target cell ratio (E:T) of 10:1. Lactate dehydrogenase (LDH) release was measured using the CytoTox-One reagent (Promega). Specific lysis was determined by normalizing the data to maximal (detergent) and background (effector and target cells alone) lysis. The mean and SD of triplicate measures using the NK cells from three donors is shown.

FIG. 6 is a graphical representation showing results of a NK cell activation assay. PBMCs from healthy donors were incubated with BTN2A1-positive target cells (LM-MEL-62) in the presence and absence of anti-BTN2A1 antibody (34C1). NK cell activation was determined by examining the proportion of NK cells (CD3⁻, CD56⁺) that were CD107a⁺by flow cytometry. The mean and SD from 4 experiments is shown.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive). Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The description and definitions of variable regions and parts thereof, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

For the purposes of nomenclature only and not limitation, the amino acid sequence of a BTN2A1 is taught in NCBI RefSeq NP_001184162.1, NP_001184163.1, NP_008980.1 or NP_001184163.1 and/or in SEQ ID NOs: 1-4. In one example, the BTN2A1 is human BTN2A1.

The term “melanoma” refers to a tumor of high malignancy that starts in melanocytes of normal skin or moles and metastasizes rapidly and widely. The term “melanoma” can be used interchangeably with the terms “malignant melanoma”, “melanocarcinoma”, “melanoepithelioma”, and “melanosarcoma”.

The term “immunoglobulin” will be understood to include any antigen binding protein comprising an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a V_{H}, however lack a V_{L} and are often referred to as heavy chain immunoglobulins. Other “immunoglobulins” include T cell receptors.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region or constant fragment or fragment crystallizable (Fc). A V_{H} and a V_{L} interact to form a Fv comprising an antigen binding region that specifically binds to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof. In some cases, the intact antibody may be capable of inducing one or more effector functions.

The term “naked antibody” refers to an antibody that is not conjugated to another compound, e.g., a toxic compound or radiolabel.

An “antigen binding fragment” of an antibody comprises one or more variable regions of an intact antibody. Examples of antibody fragments include Fab, Fab′, F(ab′)₂ and Fv fragments; diabodies; linear antibodies; single-chain antibody molecules and multispecific antibodies formed from antibody fragments.

In the context of the present disclosure, “effector functions” refer to those biological activities mediated by cells or proteins that bind to the Fc region (a native sequence Fc region or amino acid sequence variant Fc region) of an antibody that result in killing of a cell. Examples of effector functions induced by antibodies or antigen binding fragments thereof include: complement dependent cytotoxicity; antibody-dependent-cell-mediated cytotoxicity (ADCC); antibody-dependent-cell-phagocytosis (ADCP); and B-cell activation.

“Antibody-dependent-cell-mediated cytotoxicity” or “ADCC” refers to a form of cytotoxicity in which secreted Ig bound onto Fc receptors (“FcRs”) present on certain cytotoxic cells (e.g., natural killer (“NK”) cells, neutrophils and macrophages) enable these cytotoxic effector cells to bind specifically to an antigen-bearing target-cell and subsequently kill the target-cell with cytotoxins. To assess ADCC activity of a molecule of interest, an in vitro ADCC assay may be performed. Useful effector cells for such assays include peripheral blood mononuclear cells (“PBMC”) and NK cells.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”. According to the numbering system of Kabat, V_{H} FRs and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4).

“Framework regions” (hereinafter FR) are those variable domain residues other than the CDR residues.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy (C_{H})1, a linker, a C_{H}2 and a C_{H}3. In a heavy chain, a constant region comprises a Fc. In a light chain, a constant region generally comprise one constant domain (a C_{L}1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, α or δ heavy chain comprises two constant domains.

The term “EU numbering system of Kabat” will be understood to mean the numbering of an antibody heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

As used herein, the term “binds” in reference to the interaction of a compound with an antigen means that the interaction is dependent upon the presence of a particular structure (e.g., an antigenic determinant or epitope) on the antigen. For example, a compound, such as an antibody, recognizes and binds to a specific protein structure rather than to proteins generally. If a compound binds to epitope “A”, the presence of a molecule containing epitope “A” (or free, unlabeled “A”), in a reaction containing labeled “A” and the compound, will reduce the amount of labeled “A” bound to the compound.

As used herein, the term “specifically binds” shall be taken to mean that the binding interaction between an antibody or antigen binding fragment thereof and BTN2A1 chain is dependent on the presence of the antigenic determinant or epitope of an BTN2A1 chain bound by the antibody or antigen binding fragment thereof. Accordingly, the antibody or antigen binding fragment thereof preferentially binds or recognizes an BTN2A1 chain antigenic determinant or epitope even when present in a mixture of other molecules or organisms. In one example, the antibody or antigen binding fragment thereof reacts or associates more frequently, more rapidly, with greater duration and/or with greater affinity with BTN2A1 or cell expressing same than it does with alternative antigens or cells. It is also understood by reading this definition that, for example, an antibody or antigen binding fragment thereof specifically binds to BTN2A1 may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. For example, a compound of the disclosure is contacted with BTN2A1 or a cell expressing same or a mutant form thereof or an alternative antigen. The binding of the compound to the BTN2A1 or mutant form or alternative antigen is then determined and a compound that binds as set out above to the BTN2A1 rather than the mutant or alternative antigen is considered to specifically bind to BTN2A1.

As used herein, the term “neutralize” shall be taken to mean that an antibody or antigen binding fragment thereof is capable of reducing or preventing BTN2A1 signaling in a cell and/or reducing or preventing BTN2A1 binding to a ligand thereof. Methods for determining whether or not a compound neutralizes BTN2A1 signaling will be apparent to the skilled artisan based on the description herein.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease are mitigated or eliminated.

As used herein, the term “prevention” includes providing prophylaxis with respect to occurrence or recurrence of a disease in an individual. An individual may be predisposed to or at risk of developing the disease or disease relapse but has not yet been diagnosed with the disease or the relapse.

An “effective amount” refers to at least an amount effective, at dosages and for periods of time necessary, to achieve the desired therapeutic or prophylactic result. An effective amount can be provided in one or more administrations. In some examples of the present disclosure, the term “effective amount” is meant an amount necessary to effect treatment of a disease or condition as hereinbefore described. The effective amount may vary according to the disease or condition to be treated and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. The effective amount can be administered in a single dose or in a dose repeated once or several times over a treatment period.

A “therapeutically effective amount” is at least the minimum concentration required to effect a measurable improvement of a particular disorder (e.g., melanoma). A therapeutically effective amount herein may vary according to factors such as the disease state, age, sex, and weight of the patient, and the ability of the compound (e.g., antibody or antigen binding fragment thereof) to elicit a desired response in the individual. A therapeutically effective amount is also one in which any toxic or detrimental effects of the antibody or antigen binding fragment thereof are outweighed by the therapeutically beneficial effects. In the case of melanoma, the therapeutically effective amount of the compound may reduce the number of cancer cells; reduce the primary tumor size; inhibit (i.e., slow to some extent and, in some examples, stop) cancer cell infiltration into peripheral organs; inhibit (i.e., slow to some extent and, in some examples, stop) tumor metastasis; inhibit or delay, to some extent, tumor growth or tumor progression; and/or relieve to some extent one or more of the symptoms associated with the disorder. To the extent the compound may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The “mammal” treated according to the present disclosure may be a mammal, such as a non-human primate or a human. In one example, the mammal is a human.

### Conditions to be Treated

In some examples of the disclosure, a method described herein is for the treatment of a cancer. The term “cancer” refers to or describes the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but are not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In some example, cancers that are amenable to treatment with compounds of the disclosure include melanoma prostate cancer, colorectal cancer and lung cancer (e.g., non-small cell lung cancer).

In one example, methods of the disclosure treat melanoma. Melanomas predominantly occur in skin, but are also found in other parts of the body, including the bowel and the eye e.g. uveal melanoma). Melanoma can originate in any part of the body that contains melanocytes. Examples of melanoma include, but are not limited to superficial spreading melanoma, nodular melanoma, Lentigo maligna melanoma, and Acral lentiginous melanoma.

Melanoma can be staged depending on a number of criteria including size, ulceration, spread to lymph nodes, and/or spread to other tissues or organs.

In one example, the melanoma is staged according to a T category, which is based on the thickness of the melanoma and other factors seen in the skin biopsy. For example, the method the disclosure is used to treat a melanoma falling into one of the following categories:
Tis: Melanoma in situ. (The tumor remains in the epidermis, the outermost layer of skin.);
T1a: The melanoma is less than or equal to 1.0 mm thick, without ulceration and with a mitotic rate of less than 1/mm²;
T1b: The melanoma is less than or equal to 1.0 mm thick. It is ulcerated and/or the mitotic rate is equal to or greater than 1/mm²;
T2a: The melanoma is between 1.01 and 2.0 mm thick without ulceration;
T2b: The melanoma is between 1.01 and 2.0 mm thick with ulceration;
T3a: The melanoma is between 2.01 and 4.0 mm thick without ulceration;
T3b: The melanoma is between 2.01 and 4.0 mm thick with ulceration;
T4a: The melanoma is thicker than 4.0 mm without ulceration;
T4b: The melanoma is thicker than 4.0 mm with ulceration.

In the above categories, the following characteristics are considered:
Tumor thickness: thickness of the melanoma also called the Breslow measurement.
Mitotic rate: To measure the mitotic rate, a pathologist counts the number of cells in the process of dividing (mitosis) in a certain area of melanoma tissue.
Ulceration: Ulceration is a breakdown of the skin over the melanoma. Melanomas that are ulcerated tend to have a worse prognosis.

In one example, the melanoma is staged according to a N category, which is based on whether or not a sentinel lymph node biopsy was done. The clinical staging of the lymph nodes, which is done without the sentinel node biopsy, is:
NX: Nearby (regional) lymph nodes cannot be assessed.
N0: No spread to nearby lymph nodes.
N1: Spread to 1 nearby lymph node.
N2: Spread to 2 or 3 nearby lymph nodes, OR spread of melanoma to nearby skin or toward a nearby lymph node area (without reaching the lymph nodes).
N3: Spread to 4 or more lymph nodes, OR spread to lymph nodes that are clumped together, OR spread of melanoma to nearby skin or toward a lymph node area and into the lymph node(s).

Following a lymph node biopsy, the pathologic stage can be determined, and the staging is as follows:
Any Na (N1a or N2a) means that the melanoma is in the lymph node(s), but it is so small that it is only seen under the microscope (also known as microscopic spread).
Any Nb (N1b or N2b) means that the melanoma is in the lymph node(s) and was large enough to be visible on imaging tests or felt by the doctor before it was removed (also known as macroscopic spread).
N2c means the melanoma has spread to very small areas of nearby skin (satellite tumors) or has spread to skin lymphatic channels around the tumor (without reaching the lymph nodes).

In one example, the melanoma is staged according to a M category, which is based on whether or not metastases are present. M categories are as follows:
M0: No distant metastasis.
M1a: Metastasis to skin, subcutaneous (below the skin) tissue, or lymph nodes in distant parts of the body, with a normal blood lactate dehydrogenase (LDH) level.
M1b: Metastasis to the lungs, with a normal blood LDH level.
M1c: Metastasis to other organs, OR distant spread to any site along with an elevated blood LDH level.

In one example, the melanoma is staged according to a stage grouping. Once the T, N, and M groups have been determined, they are combined to give an overall stage. Stage groupings are as follows:
Stage 0: Tis, N0, M0: The melanoma is in situ, meaning that it is in the epidermis but has not spread to the dermis (lower layer).
Stage IA: T1a, N0, M0: The melanoma is less than 1.0 mm in thickness. It is not ulcerated and has a mitotic rate of less than 1/mm². It has not been found in lymph nodes or distant organs.
Stage IB: T1b or T2a, N0, M0: The melanoma is less than 1.0 mm in thickness and is ulcerated or has a mitotic rate of at least 1/mm2, OR it is between 1.01 and 2.0 mm and is not ulcerated. It has not been found in lymph nodes or distant organs.
Stage IIA: T2b or T3a, N0, M0: The melanoma is between 1.01 mm and 2.0 mm in thickness and is ulcerated, OR it is between 2.01 and 4.0 mm and is not ulcerated. It has not been found in lymph nodes or distant organs.
Stage IIB: T3b or T4a, N0, M0: The melanoma is between 2.01 mm and 4.0 mm in thickness and is ulcerated, OR it is thicker than 4.0 mm and is not ulcerated. It has not been found in lymph nodes or distant organs.
Stage IIC: T4b, N0, M0: The melanoma is thicker than 4.0 mm and is ulcerated. It has not been found in lymph nodes or distant organs.
Stage IIIA: T1a to T4a, N1a or N2a, M0: The melanoma can be of any thickness, but it is not ulcerated. It has spread to 1 to 3 lymph nodes near the affected skin area, but the nodes are not enlarged and the melanoma is found only when they are viewed under the microscope. There is no distant spread.
Stage IIIB: One of the following applies:
   T1b to T4b, N1a or N2a, M0: The melanoma can be of any thickness and is ulcerated. It has spread to 1 to 3 lymph nodes near the affected skin area, but the nodes are not enlarged and the melanoma is found only when they are viewed under the microscope. There is no distant spread.
   T1a to T4a, N1b or N2b, M0: The melanoma can be of any thickness, but it is not ulcerated. It has spread to 1 to 3 lymph nodes near the affected skin area. The nodes are enlarged because of the melanoma. There is no distant spread.
   T1a to T4a, N2c, M0: The melanoma can be of any thickness, but it is not ulcerated. It has spread to small areas of nearby skin or lymphatic channels around the original tumor, but the nodes do not contain melanoma. There is no distant spread.
Stage IIIC: One of the following applies:
   T1b to T4b, N1b or N2b, M0: The melanoma can be of any thickness and is ulcerated. It has spread to 1 to 3 lymph nodes near the affected skin area. The nodes are enlarged because of the melanoma. There is no distant spread.
   T1b to T4b, N2c, M0: The melanoma can be of any thickness and is ulcerated. It has spread to small areas of nearby skin or lymphatic channels around the original tumor, but the nodes do not contain melanoma. There is no distant spread.
   Any T, N3, M0: The melanoma can be of any thickness and may or may not be ulcerated. It has spread to 4 or more nearby lymph nodes, OR to nearby lymph nodes that are clumped together, OR it has spread to nearby skin or lymphatic channels around the original tumor and to nearby lymph nodes. The nodes are enlarged because of the melanoma. There is no distant spread.
Stage IV: Any T, any N, M1(a, b, or c): The melanoma has spread beyond the original area of skin and nearby lymph nodes to other organs such as the lung, liver, or brain, or to distant areas of the skin, subcutaneous tissue, or distant lymph nodes. Neither spread to nearby lymph nodes nor thickness is considered in this stage, but typically the melanoma is thick and has also spread to the lymph nodes.

In one example, the disclosure provides methods of treating a Stage 0 melanoma.

In one example, the disclosure provides methods of treating a Stage I melanoma (e.g., stage IA or stage IB).

In one example, the disclosure provides methods of treating a Stage II melanoma (e.g., stage IIA, stage IIB or stage ITC).

In one example, the disclosure provides methods of treating a Stage III melanoma (e.g., stage IIIA, stage IIIB or stage IIIC).

In one example, the disclosure provides methods of treating a Stage IV melanoma.

### Compounds

As discussed herein, compounds of the present disclosure can take various forms, e.g., protein-based compounds or chemical compounds. Typically, the compounds are antibodies or antigen binding fragments thereof. Exemplary compounds are discussed herein.

### Antibodies

### Immunization-Based Methods

Methods for generating antibodies are known in the art and/or described in Harlow and Lane (editors) Antibodies: A Laboratory Manual, Cold Spring Harbor Laboratory, (1988). Generally, in such methods an BTN2A1 protein or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

Monoclonal antibodies are one exemplary form of antibody contemplated by the present disclosure. The term “monoclonal antibody” or “MAb” refers to a homogeneous antibody population capable of binding to the same antigen(s), for example, to the same epitope within the antigen. This term is not intended to be limited as regards to the source of the antibody or the manner in which it is made.

For the production of Mabs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human immunoglobulin proteins and, for example, do not express murine immunoglobulin proteins, can also be used to generate an antibody of the present disclosure (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

### Library-Based Methods

The present disclosure also encompasses screening of libraries of antibodies or antigen binding fragments thereof (e.g., comprising variable regions thereof).

Examples of libraries contemplated by this disclosure include naïve libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3rd Ed, vols. 1-3, Cold Spring Harbor Laboratory Press, 2001) and used to encode and display proteins using a method known in the art. Other techniques for producing libraries of proteins are described in, for example in U.S. Pat. No. 6,300,064 (e.g., a HuCAL library of Morphosys AG); U.S. Pat. No. 5,885,793; U.S. Pat. No. 6,204,023; U.S. Pat. No. 6,291,158; or U.S. Pat. No. 6,248,516.

The antigen binding fragments according to the disclosure may be soluble secreted proteins or may be presented as a fusion protein on the surface of a cell, or particle (e.g., a phage or other virus, a ribosome or a spore). Various display library formats are known in the art. For example, the library is an in vitro display library (e.g., a ribosome display library, a covalent display library or a mRNA display library, e.g., as described in U.S. Pat. No. 7,270,969). In yet another example, the display library is a phage display library wherein proteins comprising antigen binding fragments of antibodies are expressed on phage, e.g., as described in U.S. Pat. No. 6,300,064; U.S. Pat. No. 5,885,793; U.S. Pat. No. 6,204,023; U.S. Pat. No. 6,291,158; or U.S. Pat. No. 6,248,516. Other phage display methods are known in the art and are contemplated by the present disclosure. Similarly, methods of cell display are contemplated by the disclosure, e.g., bacterial display libraries, e.g., as described in U.S. Pat. No. 5,516,637; yeast display libraries, e.g., as described in U.S. Pat. No. 6,423,538 or a mammalian display library.

Methods for screening display libraries are known in the art. In one example, a display library of the present disclosure is screened using affinity purification, e.g., as described in Scopes (In: Protein purification: principles and practice, Third Edition, Springer Verlag, 1994). Methods of affinity purification typically involve contacting proteins comprising antigen binding fragments displayed by the library with a target antigen (e.g., BTN2A1) and, following washing, eluting those domains that remain bound to the antigen.

Any variable regions or scFvs identified by screening are readily modified into a complete antibody, if desired. Exemplary methods for modifying or reformatting variable regions or scFvs into a complete antibody are described, for example, in Jones et al., J Immunol Methods. 354:85-90, 2010; or Jostock et al., J Immunol Methods, 289: 65-80, 2004; or WO2012/040793. Alternatively, or additionally, standard cloning methods are used, e.g., as described in Ausubel et al (In: Current Protocols in Molecular Biology. Wiley Interscience, ISBN 047 150338, 1987), and/or (Sambrook et al (In: Molecular Cloning: Molecular Cloning: A Laboratory Manual, Cold Spring Harbor Laboratories, New York, Third Edition 2001).

### Deimmunized, Chimeric, Humanized, Synhumanized, Primatized and Human Antibodies or Antigen Binding Fragments

The antibodies or antigen binding fragments of the present disclosure may be may be humanized.

The term “humanized antibody” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized antibodies also include antibodies in which one or more residues of the human protein are modified by one or more amino acid substitutions and/or one or more FR residues of the human antibody are replaced by corresponding non-human residues. Humanized antibodies may also comprise residues which are found in neither the human antibody or in the non-human antibody. Any additional regions of the antibody (e.g., Fc region) are generally human. Humanization can be performed using a method known in the art, e.g., U.S. Pat. No. 5,225,539, U.S. Pat. No. 6,054,297, U.S. Pat. No. 7,566,771 or U.S. Pat. No. 5,585,089. The term “humanized antibody” also encompasses a super-humanized antibody, e.g., as described in U.S. Pat. No. 7,732,578. A similar meaning will be taken to apply to the term “humanized antigen binding fragment”.

The antibodies or antigen binding fragments thereof of the present disclosure may be human antibodies or antigen binding fragments thereof. The term “human antibody” as used herein refers to antibodies having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human antibody will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516. A similar meaning will be taken to apply to the term “human antigen binding fragment”.

The antibodies or antigen binding fragments thereof of the present disclosure may be synhumanized antibodies or antigen binding fragments thereof. The term “synhumanized antibody” refers to an antibody prepared by a method described in WO2007/019620. A synhumanized antibody includes a variable region of an antibody, wherein the variable region comprises FRs from a New World primate antibody variable region and CDRs from a non-New World primate antibody variable region.

The antibody or antigen binding fragment thereof of the present disclosure may be primatized. A “primatized antibody” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example an antibody or antigen binding fragment thereof of the disclosure is a chimeric antibody or fragment. The term “chimeric antibody” or “chimeric antigen binding fragment” refers to an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass. In one example, a chimeric antibody comprising a V_{H} and/or a V_{L} from a non-human antibody (e.g., a murine antibody) and the remaining regions of the antibody are from a human antibody. The production of such chimeric antibodies and antigen binding fragments thereof is known in the art, and may be achieved by standard means (as described, e.g., in U.S. Pat. No. 6,331,415; U.S. Pat. No. 5,807,715; U.S. Pat. No. 4,816,567 and U.S. Pat. No. 4,816,397).

The present disclosure also contemplates a deimmunized antibody or antigen binding fragment thereof, e.g., as described in WO2000/34317 and WO2004/108158. De-immunized antibodies and fragments have one or more epitopes, e.g., B cell epitopes or T cell epitopes removed (i.e., mutated) to thereby reduce the likelihood that a subject will raise an immune response against the antibody or protein. For example, an antibody of the disclosure is analyzed to identify one or more B or T cell epitopes and one or more amino acid residues within the epitope is mutated to thereby reduce the immunogenicity of the antibody.

### Antibody Fragments

### Single-Domain Antibodies

In some examples, an antigen binding fragment of an antibody of the disclosure is or comprises a single-domain antibody (which is used interchangeably with the term “domain antibody” or “dAb”). A single-domain antibody is a single polypeptide chain comprising all or a portion of the heavy chain variable domain of an antibody.

### Diabodies, Triabodies, Tetrabodies

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

A diabody, triabody, tetrabody, etc capable of inducing effector activity can be produced using an antigen binding fragment capable of binding to BTN2A1 and an antigen binding fragment capable of binding to a cell surface molecule on an immune cell, e.g., a T cell (e.g., CD3).

Single Chain Fv (scFv) Fragments

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

The present disclosure also contemplates a dimeric scFv capable of inducing effector activity (e.g., a bispecific T cell effector, or BiTe). For example, one scFv binds to BTN2A1 and comprises CDRs and/or variable regions described herein and another scFv binds to a cell surface molecule on an immune cell, e.g., a T cell (e.g., CD3) or a NK cell (e.g., CD16 or CD16a). In one example, the dimeric protein is a combination of a dAb and a scFv. Examples of bispecific antibody fragments capable of inducing effector function are described, for example, in U.S. Pat. No. 7,235,641, WO2004/106380 and Stein et al., Antibodies, 1: 88-123, 2012).

### Other Antibodies and Antibody Fragments

The present disclosure also contemplates other antibodies and antibody fragments, such as:

(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### Immunoglobulins and Immunoglobulin Fragments

An example of a compound of the present disclosure is a protein (e.g., an antibody mimetic) comprising a variable region of an immunoglobulin, such as a T cell receptor or a heavy chain immunoglobulin (e.g., an IgNAR, a camelid antibody).

### Heavy Chain Immunoglobulins

Heavy chain immunoglobulins differ structurally from many other forms of immunoglobulin (e.g., antibodies), in so far as they comprise a heavy chain, but do not comprise a light chain. Accordingly, these immunoglobulins are also referred to as “heavy chain only antibodies”. Heavy chain immunoglobulins are found in, for example, camelids and cartilaginous fish (also called IgNAR).

The variable regions present in naturally occurring heavy chain immunoglobulins are generally referred to as “Vim domains” in camelid Ig and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

Heavy chain immunoglobulins do not require the presence of light chains to bind with high affinity and with high specificity to a relevant antigen. This means that single domain binding fragments can be derived from heavy chain immunoglobulins, which are easy to express and are generally stable and soluble.

A general description of heavy chain immunoglobulins from camelids and the variable regions thereof and methods for their production and/or isolation and/or use is found inter alia in the following references WO94/04678, WO97/49805 and WO 97/49805.

A general description of heavy chain immunoglobulins from cartilaginous fish and the variable regions thereof and methods for their production and/or isolation and/or use is found inter alia in WO2005/118629.

### V-Like Proteins

An example of a compound of the disclosure is a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

### Adnectins

In one example, a compound of the disclosure is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

### Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

### Affibodies

In a further example, a compound of the disclosure is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

### Avimers

In a further example, a compound of the disclosure is an Avimer. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see WO2002088171.

### DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### Other Non Antibody Polypeptides

Other non-antibody proteins comprising binding domains include those based on human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins).

### Soluble BTN2A1

Other proteins that can neutralize BTN2A1 include mutant BTN2A1 proteins and secreted proteins comprising at least part of the extracellular portion of BTN2A1. For example, a soluble BTN2A1 can be prepared by fusing an or all extracellular domains of BTN2A1 with a Fc region of an antibody (e.g., an IgG1 antibody) or with a hinge region and a Fc region of an antibody (e.g., an IgG1 antibody).

### Constant Regions

The present disclosure encompasses compounds (e.g., antibodies and antigen binding fragments thereof) comprising a constant region of an antibody and/or a Fc region of an antibody.

Sequences of constant regions and/or Fc regions useful for producing the immunoglobulins, antibodies or antigen binding fragments of the present disclosure may be obtained from a number of different sources. In some examples, the constant region, Fc or portion thereof of the compound is derived from a human antibody. The constant region, Fc or portion thereof may be derived from any antibody class, including IgA, IgM, IgG, IgD, IgA and IgE, and any antibody isotype, including IgG1, IgG2, IgG3 and IgG4. In one example, the constant region or Fc is human isotype IgG1 or human isotype IgG2 or human isotype IgG3 or a hybrid of any of the foregoing.

In one example, the constant region or Fc region is capable of inducing an effector function. For example, the constant region or Fc region is a human IgG1 or IgG3 Fc region. In another example, the constant region or Fc region is a hybrid of an IgG1 and an IgG2 constant region or Fc region or a hybrid of an IgG1 and an IgG3 constant region or Fc region or a hybrid of an IgG2 and an IgG3 constant region or Fc region. Exemplary hybrids of human IgG1 and IgG2 constant region or Fc regions are described in Chappel et al., Proc. Natl Acad. Sci. USA, 88: 9036-9040, 1991.

Methods for determining whether or not a Fc region can induce effector function will be apparent to the skilled artisan and/or described herein.

### Effector Function

In one example, a compound (e.g., an antibody or antigen binding fragment thereof) of the present disclosure comprises an antibody Fc region capable of inducing an effector function. For example, the effector function is Fc-mediated effector function. In one example, the Fc region is an IgG1 Fc region or an IgG3 Fc region or a hybrid IgG1/IgG2 Fc region.

Suitably, a compound of the disclosure (e.g., an anti-BTN2A1 antibody or antigen binding fragment thereof) has or displays an effector function that facilitates or enables killing or at least partial depletion, substantial depletion or elimination of BTN2A1 expressing cells. Such an effector function may be enhanced binding affinity to Fc receptors, antibody-dependent cell-mediated cytotoxicity (ADCC), antibody-dependent cell mediated phagocytosis (ADCP) and/or complement dependent cytotoxicity (CDC).

In one example, a compound (e.g., an antibody or antigen binding fragment thereof) of the present disclosure is capable of inducing a similar (e.g., not significantly different or within about 10%) or the same level of effector function as a wild-type human IgG1 and/or human IgG3 Fc region.

In one example, the compound is capable of inducing an enhanced level of effector function.

In one example, the level of effector function induced by a compound comprising an Fc region is enhanced relative to that of the compound when it comprises a wild-type IgG1 Fc region

In one example, the level of effector function induced by an antibody or antigen binding fragment thereof of the disclosure is enhanced relative to that of the antibody or antigen binding fragment thereof when it comprises a wild-type IgG1 Fc region.

For the IgG class of antibodies, some effector functions (e.g., ADCC and ADCP) are governed by engagement of the Fc region with a family of receptors referred to as the Fcγ receptors (FcγRs) which are expressed on a variety of immune cells and/or with complement, e.g., C1q (e.g., CDC).

Formation of the Fc/FcγR complex recruits immune cells to sites of bound antigen, typically resulting in signaling and subsequent immune responses. Methods for optimizing the binding affinity of the FcγRs to the antibody Fc region in order to enhance the effector functions, e.g., to alter the ADCC activity relative to the “parent” Fc region, are known to persons skilled in the art. These methods can include modification of the Fc region of the antibody to enhance its interaction with relevant Fc receptors and increase its potential to facilitate ADCC and ADCP. Enhancements in ADCC activity have also been described following the modification of the oligosaccharide covalently attached to IgG1 antibodies at the conserved Asn297 in the Fc region.

It will be appreciated by the skilled artisan that in some non-limiting examples, enhancing effector function such as ADCC may be achieved by modification of a compound (e.g., an antibody) which has a normally glycosylated wild-type constant domain, including alteration or removal of glycosylation (see for example WO00/61739) and/or amino acid sequence mutations (see for example WO2008036688).

In one example, the compound binds to BTN2A1 in such a manner that it is capable of inducing an effector function, such as, ADCC.

In one example, the compound binds to an epitope within BTN2A1 that permits it to induce an effector function, such as ADCC.

In another example, the compound is capable of binding to BN2A1 on a cell in a mammal to thereby induce an effector function, such as ADCC.

For example, the compound remains bound to BTN2A1 on the surface of a cell for a time sufficient to induce an effector function, such as ADCC. For example, the compound is not internalized too quickly to permit ADCC to be induced.

Alternatively, or in addition, the compound is bound to the BTN2A1 on the surface of the cell in a manner permitting an immune effector cell to bind to a constant region or Fc region in the compound and induce an effector function, such as ADCC. For example, the Fc region of the compound is exposed in such a manner when the compound is bound to the BTN2A1 that is capable of interacting with a Fc receptor (e.g., a FcγR) on an immune effector cell. In the context of the present disclosure, the term “immune effector cell” shall be understood to mean any cell that expresses a Fc receptor and that is capable of killing a cell to which it is bound by ADCC or ADCP. In one example, the immune effector cell is a NK cell.

Each of the above paragraphs relating to effector functions of an antibody or antigen binding fragment shall be taken to apply mutatis mutandis to inducing CDC. For example, the compound is bound to the BTN2A1 on the surface of the cell in a manner permitting complement component C1q to bind to a constant region or Fc region in the compound and induce CDC.

Moreover, each of the above paragraphs relating to effector functions of an antibody or antigen binding fragment shall be taken to apply mutatis mutandis to inducing cell-mediated effector function (e.g. ADCC and/or ADCP) by virtue of a compound other than a Fc region or constant region of an antibody. For example, the cell-mediated effector function is elicited using a compound that binds to BTN2A1 as described herein and to an immune effector cells (e.g., by virtue of binding to CD16 on NK cells and neutrophils and/or CD4 on T cells).

The skilled addressee will appreciate that greater effector function may be manifested in any of a number of ways, for example as a greater level of effect, a more sustained effect or a faster rate of effect.

In one example, the constant region or Fc region comprises one or more amino acid modifications that increase its ability to induce enhanced effector function. In one example, the constant region or Fc region binds with greater affinity to one or more FcγRs. In one example, the constant region or Fc region has an affinity for an FcγR that is more than 1-fold greater than that of a wild-type constant region or Fc region or more than 5-fold greater than that of a wild-type constant region or Fc region or between 5-fold and 300-fold greater than that of a wild-type constant region or Fc region. In one example, the constant region or Fc region comprises at least one amino acid substitution at a position selected from the group consisting of: 230, 233, 234, 235, 239, 240, 243, 264, 266, 272, 274, 275, 276, 278, 302, 318, 324, 325, 326, 328, 330, 332, and 335, numbered according to the EU index of Kabat. In one example, the constant region or Fc region comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y, numbered according to the EU index of Kabat. In one example, the constant region or Fc region comprises amino acid substitutions selected from the group consisting of V264I, F243L/V264I, L328M, I332E, L328M/I332E, V264I/I332E, S298A/I332E, S239E/I332E, S239Q/I332E, S239E, A330Y, I332D, L328I/I332E, L328Q/I332E, V264T, V240I, V266I, S239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, numbered according to the EU index of Kabat.

In another example, the constant region or Fc region binds to FcγRIIIa more efficiently than to FcγRIIb. For example, the constant region or Fc region comprises at least one amino acid substitution at a position selected from the group consisting of: 234, 235, 239, 240, 264, 296, 330, and I332, numbered according to the EU index of Kabat. In one example, the constant region or Fc region comprises at least one amino acid substitution selected from the group consisting of: L234Y, L234I, L235I, S239D, S239E, S239N, S239Q, V240A, V240M, V264I, V264Y, Y296Q, A330L, A330Y, A330I, I332D, and I332E, numbered according to the EU index of Kabat. For example, the constant region or Fc region comprises amino acid substitutions selected from the group consisting of: I332E, V264I/I332E, S239E/I332E, S239Q/I332E, Y296Q, A330L, A330Y, I332D, S239D, S239D/I332E, A330Y/I332E, V264I/A330Y/I332E, A330L/I332E, V264I/A330L/I332E, L234Y, L234I, L235I, V240A, V240M, V264Y, A330I, S239D/A330L/I332E, S239D/S298A/I332E, S239N/S298A/I332E, S239D/V264I/I332E, S239D/V264I/S298A/I332E, and S239D/V264I/A330L/I332E, numbered according to the EU index of Kabat.

In a further example, the constant region or Fc region induces ADCC at a level greater than that mediated by a wild-type constant region or Fc region. For example, the constant region or Fc region induces ADCC at a level that is more than 5-fold or between 5-fold and 1000-fold greater than that induced by a wild-type constant region or Fc region. In one example, the constant region or Fc region comprises at least one amino acid substitution at a position selected from the group consisting of: 230, 233, 234, 235, 239, 240, 243, 264, 266, 272, 274, 275, 276, 278, 302, 318, 324, 325, 326, 328, 330, 332, and 335, numbered according to the EU index of Kabat. In one example, the constant region or Fc region comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y, numbered according to the EU index of Kabat. In one example, the constant region or Fc region comprises amino acid substitutions selected from the group consisting of: V264I, F243L/V264I, L328M, I332E, L328M/I332E, V264I/I332E, S298A/I332E, S239E/I332E, S239Q/I332E, S239E, A330Y, I332D, L328I/I332E, L328Q/I332E, V264T, V240I, V266I, S239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, numbered according to the EU index of Kabat.

In one example, the constant region or Fc region comprises the following amino acid substitutions S239D/I332E, numbered according to the EU index of Kabat. This constant region or Fc region has about 14 fold increase in affinity for FcγRIIIa compared to a wild-type constant region or Fc region and about 3.3 increased ability to induce ADCC compared to a wild-type constant region or Fc region.

In one example, the constant region or Fc region comprises the following amino acid substitutions S239D/A330L/I332E, numbered according to the EU index of Kabat. This constant region or Fc region has about 138 fold increase in affinity for FcγRIIIa compared to a wild-type constant region or Fc region and about 323 increased ability to induce ADCC compared to a wild-type constant region or Fc region.

Additional amino acid substitutions that increase ability of a Fc region to induce effector function are known in the art and/or described, for example, in U.S. Pat. No. 6,737,056 or U.S. Pat. No. 7,317,091.

In one example, the glycosylation of the constant region or Fc region is altered to increase its ability to induce enhanced effector function. In this regard, native antibodies produced by mammalian cells typically comprise a branched, biantennary oligosaccharide that is generally attached by an N-linkage to Asn297 of the C_{H}2 domain of the constant region or Fc region. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some examples, constant regions or Fc regions according to the present disclosure comprise a carbohydrate structure that lacks fucose attached (directly or indirectly) to an Fc region, i.e., the Fc region is “afucosylated”. Such variants may have an improved ability to induce ADCC. Methods for producing afucosylated Fc regions or constant regions include, expressing the immunoglobulin or antibody in a cell line incapable of expressing α-1,6-fucosyltransferase (FUT8) (e.g., as described in Yumane-Ohnuki et al., Biotechnol. Bioengineer., 87: 614-622, 2004), expressing the immunoglobulin or antibody in cells expressing a small interfering RNA against FUT8 (e.g., as described in Mori et al., Biotechnol. Bioengineer., 88: 901-908, 2004), expressing the antibody or antigen binding fragment in cells incapable of expressing guanosine diphosphate (GDP)-mannose 4,6-dehydratase (GMD) (e.g., as described in Kanda et al., J. Biotechnol., 130: 300-310, 2007). The present disclosure also contemplates the use of compounds having a reduced level of fucosylation, e.g., produced using a cell line modified to express β-(1,4)-N-acetylglucosaminyltransferase III (GnT-III) (e.g., as described in Umāna et al., Nat. Biotechnol., 17: 176-180, 1999).

In one example, an antibody or antigen binding fragment according to the present disclosure is afucosylated. For example, the immunoglobulin or antibody is produced in a cell (e.g., a mammalian cell, such as a CHO cell) that does not express FUT8.

Other methods include the use of cell lines which inherently produce Fc regions or constant regions or antigen binding fragments capable of inducing enhanced Fc-mediated effector function (e.g. duck embryonic derived stem cells for the production of viral vaccines, WO2008/129058; Recombinant protein production in avian EBX® cells, WO2008/142124).

Compounds (e.g., antibodies or antigen binding fragments) useful in the methods of the present disclosure also include those with bisected oligosaccharides, e.g., in which a biantennary oligosaccharide attached to the constant region or Fc region is bisected by GlcNAc. Such compounds may have reduced fucosylation and/or improved ADCC function. Examples of such compounds are described, e.g., in U.S. Pat. No. 6,602,684 and US20050123546.

Compounds (e.g., antibodies or antigen binding fragments) with at least one galactose residue in the oligosaccharide attached to the constant region or Fc region are also contemplated. Such antibodies or antigen binding fragments may have improved CDC function. Such immunoglobulins are described, e.g., in WO1997/30087 and WO1999/22764.

Methods for determining the ability of a compound to induce effector function and known in the art and/or described in more detail herein.

### Stabilized Proteins

Neutralizing proteins of the present disclosure can comprise an IgG4 constant region or a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

### Additional Modifications

The present disclosure also contemplates additional modifications to constant regions or Fc regions of compounds (e.g., antibodies or antigen binding fragments).

For example, constant region of Fc region comprises one or more amino acid substitutions that increase the half-life of the antibody or fragment. For example, the constant region or Fc region comprises one or more amino acid substitutions that increase the affinity of the constant region or Fc region for the neonatal Fc region (FcRn). For example, the constant region or Fc region has increased affinity for FcRn at lower pH, e.g., about pH 6.0, to facilitate Fc/FcRn binding in an endosome. In one example, the constant region or Fc region has increased affinity for FcRn at about pH 6 compared to its affinity at about pH 7.4, which facilitates the re-release of constant region or Fc into blood following cellular recycling. These amino acid substitutions are useful for extending the half-life of a Fc containing or constant region containing compound, by reducing clearance from the blood.

Exemplary amino acid substitutions include T250Q and/or M428L according to the EU numbering system of Kabat. Additional or alternative amino acid substitutions are described, for example, in US20070135620.

### Peptides

In one example, a compound is a peptide, e.g., isolated from a random peptide library. To identify a suitable peptide, a random peptide library is generated and screened as described in U.S. Pat. No. 5,733,731, U.S. Pat. No. 5,591,646 and U.S. Pat. No. 5,834,318. Generally, such libraries are generated from short random oligonucleotides that are expressed either in vitro or in vivo and displayed in such a way to facilitate screening of the library to identify a peptide that is capable of specifically binding to BTN2A1. Methods of display include, phage display, retroviral display, bacterial surface display, bacterial flagellar display, bacterial spore display, yeast surface display, mammalian surface display, and methods of in vitro display including, mRNA display, ribosome display and covalent display.

A peptide that is capable of binding to BTN2A1 is identified by any of a number of methods known in the art, such as, for example, standard affinity purification methods as described, for example in Scopes, 1994) purification using FACS analysis as described in US645563.

### Small Molecules

In another example, a compound is a small molecule. Such a small molecule may be isolated from a library. Chemical small molecule libraries are available commercially or alternatively may be generated using methods known in the art, such as, for example, those described in U.S. Pat. No. 5,463,564.

Techniques for synthesizing small organic compounds will vary considerably depending upon the compound, however such methods will be known to those skilled in the art.

In one example, informatics is used to select suitable chemical building blocks from known compounds, for producing a combinatorial library. For example, QSAR (Quantitative Structure Activity Relationship) modeling approach uses linear regressions or regression trees of compound structures to determine suitability. The software of the Chemical Computing Group, Inc. (Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and log P (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, lead compounds identified in initial screens, can be used to expand the list of compounds being screened to thereby identify highly active compounds.

### Nucleic Acid Aptamers

In another example, a compound is a nucleic acid aptamer (adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., BTN2A1. Thus, aptamers are the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can be prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to BTN2A1) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. No. 5,270,163; and/or U.S. Pat. No. 5,475,096.

### Nucleic Acid-Based BTN2A1 Signaling Inhibitors

In one example of the disclosure, therapeutic methods as described herein according to any example of the disclosure involve reducing expression of BTN2A1. For example, such a method involves administering a compound that reduces transcription and/or translation of the nucleic acid. In one example, the compound is a nucleic acid, e.g., an antisense polynucleotide, a ribozyme, a PNA, an interfering RNA, a siRNA, a microRNA

### Antisense Nucleic Acids

The term “antisense nucleic acid” shall be taken to mean a DNA or RNA or derivative thereof (e.g., LNA or PNA), or combination thereof that is complementary to at least a portion of a specific mRNA molecule encoding a polypeptide as described herein in any example of the disclosure and capable of interfering with a post-transcriptional event such as mRNA translation. The use of antisense methods is known in the art (see for example, Hartmann and Endres (editors), Manual of Antisense Methodology, Kluwer (1999)).

An antisense nucleic acid of the disclosure will hybridize to a target nucleic acid under physiological conditions. Antisense nucleic acids include sequences that correspond to structural genes or coding regions or to sequences that effect control over gene expression or splicing. For example, the antisense nucleic acid may correspond to the targeted coding region of a nucleic acid encoding BTN2A1, or the 5′-untranslated region (UTR) or the 3′-UTR or combination of these. It may be complementary in part to intron sequences, which may be spliced out during or after transcription, for example only to exon sequences of the target gene. The length of the antisense sequence should be at least 19 contiguous nucleotides, for example, at least 50 nucleotides, such as at least 100, 200, 500 or 1000 nucleotides of a nucleic acid encoding BTN2A1. The full-length sequence complementary to the entire gene transcript may be used. The length can be 100-2000 nucleotides. The degree of identity of the antisense sequence to the targeted transcript should be at least 90%, for example, 95-100%.

### Catalytic Nucleic Acid

The term “catalytic nucleic acid” refers to a DNA molecule or DNA-containing molecule (also known in the art as a “deoxyribozyme” or “DNAzyme”) or a RNA or RNA-containing molecule (also known as a “ribozyme” or “RNAzyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are useful in this disclosure are a hammerhead ribozyme and a hairpin ribozyme.

### RNA Interference

RNA interference (RNAi) is useful for specifically inhibiting the production of a particular protein. Without being limited by theory, this technology relies on the presence of dsRNA molecules that contain a sequence that is essentially identical to the mRNA of the gene of interest or part thereof, in this case an mRNA encoding a BTN2A1. Conveniently, the dsRNA can be produced from a single promoter in a recombinant vector host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present disclosure is well within the capacity of a person skilled in the art, particularly considering WO99/32619, WO99/53050, WO99/49029, and WO01/34815.

The length of the sense and antisense sequences that hybridize should each be at least 19 contiguous nucleotides, such as at least 30 or 50 nucleotides, for example at least 100, 200, 500 or 1000 nucleotides. The full-length sequence corresponding to the entire gene transcript may be used. The lengths can be 100-2000 nucleotides. The degree of identity of the sense and antisense sequences to the targeted transcript should be at least 85%, for example, at least 90% such as, 95-100%.

Exemplary small interfering RNA (“siRNA”) molecules comprise a nucleotide sequence that is identical to about 19-21 contiguous nucleotides of the target mRNA. For example, the siRNA sequence commences with the dinucleotide AA, comprises a GC-content of about 30-70% (for example, 30-60%, such as 40-60% for example about 45%-55%), and does not have a high percentage identity to any nucleotide sequence other than the target in the genome of the mammal in which it is to be introduced, for example as determined by standard BLAST search. siRNA that reduce expression of BTN2A1 are commercially available from LifeTechnologies or Santa Cruz Biotechnology.

Short hairpin RNA (shRNA) that reduce expression of BTN2A1 are commercially available from, for example, Origene Technologies, Inc.

### Protein Production

### Recombinant Expression

In one example, a compound as described herein is a peptide or polypeptide (e.g., is an antibody or antigen binding fragment thereof). In one example, the compound is recombinant.

In the case of a recombinant peptide or polypeptide, nucleic acid encoding same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce immunoglobulin or antibody protein.

Exemplary cells used for expressing a peptide or polypeptide are CHO cells, myeloma cells or HEK cells. The cell may further comprise one or more genetic mutations and/or deletions that facilitate expression of a peptide or polypeptide (e.g., antibody or antigen binding fragment thereof). One non-limiting example is a deletion of a gene encoding an enzyme required for fucosylation of an expressed peptide or polypeptide (e.g., comprising a Fc region of an antibody). For example, the deleted gene encodes FUT8. A commercially available source of FUT8-deleted CHO cells is Biowa (Potelligent™ cells). For example, the cells used for expression of an afucosylated peptide or polypeptide are FUT8-deleted CHO cells, such as, Biowa's Potelligent™ cells.

Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells. Thus, another example of the disclosure provides an expression construct that comprises an isolated nucleic acid of the disclosure and one or more additional nucleotide sequences. Suitably, the expression construct is in the form of, or comprises genetic components of, a plasmid, bacteriophage, a cosmid, a yeast or bacterial artificial chromosome as are understood in the art. Expression constructs may be suitable for maintenance and propagation of the isolated nucleic acid in bacteria or other host cells, for manipulation by recombinant DNA technology and/or for expression of the nucleic acid or a compound of the disclosure.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding the compound (e.g., derived from the information provided herein), an enhancer element, a promoter, and a transcription termination sequence. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin or antibody promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the compound (e.g., antibody or antigen binding fragment) may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

The skilled artisan will understand from the foregoing description that the present disclosure also provides an isolated nucleic acid encoding a compound (e.g., a peptide or polypeptide compound or an antibody or antigen binding fragment thereof) of the present disclosure.

The present disclosure also provides an expression construct comprising an isolated nucleic acid of the disclosure operably linked to a promoter. In one example, the expression construct is an expression vector.

In one example, the expression construct of the disclosure comprises a nucleic acid encoding a polypeptide (e.g., comprising a V_{H}) operably linked to a promoter and a nucleic acid encoding another polypeptide (e.g., comprising a V_{L}) operably linked to a promoter.

In another example, the expression construct is a bicistronic expression construct, e.g., comprising the following operably linked components in 5′ to 3′ order:

(i) a promoter
(ii) a nucleic acid encoding a first polypeptide;
(iii) an internal ribosome entry site; and
(iv) a nucleic acid encoding a second polypeptide.

For example, the first polypeptide comprises a V_{H} and the second polypeptide comprises a V_{L}, or the first polypeptide comprises a V_{L} and the second polypeptide comprises a V_{H}.

The present disclosure also contemplates separate expression constructs one of which encodes a first polypeptide (e.g., comprising a V_{H}) and another of which encodes a second polypeptide (e.g., comprising a V_{L}). For example, the present disclosure also provides a composition comprising:

(i) a first expression construct comprising a nucleic acid encoding a polypeptide (e.g., comprising a V_{H}) operably linked to a promoter; and
(ii) a second expression construct comprising a nucleic acid encoding a polypeptide (e.g., comprising a V_{L}) operably linked to a promoter.

The disclosure also provides a host cell comprising an expression construct according to the present disclosure.

The present disclosure also provides an isolated cell expressing a compound (e.g., a peptide or polypeptide compound or an antibody or antigen binding fragment thereof of the disclosure or a recombinant cell genetically-modified to express the compound.

In one example, the cell comprises the expression construct of the disclosure or:

(i) a first genetic construct comprising a nucleic acid encoding a polypeptide (e.g., comprising a V_{H}) operably linked to a promoter; and
(ii) a second genetic construct comprising a nucleic acid encoding a polypeptide (e.g., comprising a V_{L}) operably linked to a promoter,
wherein the first and second polypeptides form an antibody or antigen binding fragment of the present disclosure.

The genetic construct can be integrated into the cell or remain episomal.

Examples of cells of the present disclosure include bacterial cells, yeast cells, insect cells or mammalian cells.

The present disclosure additionally provides a method for producing a compound (e.g., a peptide or polypeptide compound or an antibody or antigen binding fragment thereof) of the disclosure, the method comprising maintaining the genetic construct(s) of the disclosure under conditions sufficient for the compound to be produced.

In one example, the method for producing a compound of the disclosure comprises culturing the cell of the disclosure under conditions sufficient for the compound to be produced and, optionally, secreted.

In one example, the method for producing a compound of the disclosure additionally comprises isolating the compound thereof.

In one example, a method for producing a compound of the disclosure additionally comprises formulating the compound with a pharmaceutically acceptable carrier.

### Isolation of Proteins

Methods for purifying a peptide or polypeptide (e.g., an antibody or antigen binding fragment) are known in the art and/or described herein.

Where a peptide or polypeptide is secreted into the medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The peptide or polypeptide prepared from cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

### Peptide Synthesis

A peptide is synthesized using a chemical method known to the skilled artisan. For example, synthetic peptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

Generally, chemical synthesis methods comprise the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do notracemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B.Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis. Synthesis. Biology, Vol. 1, for classical solution synthesis. These methods are suitable for synthesis of a peptide of the present disclosure.

A peptide as described herein can also be chemically prepared by other methods such as by the method of simultaneous multiple peptide synthesis. See, e. g., Houghten Proc. Natl. Acad. Sci. USA 82: 5131-5135, 1985 or U.S. Pat. No. 4,631,211.

### Nucleic Acid Synthesis

Methods for producing/synthesizing nucleic acid-based compounds of the disclosure|are known in the art. For example, oligonucleotide synthesis is described, in Gait (editor) Oligonucleotide Synthesis: A Practical Approach, IRL Press, Oxford (1984). For example, a probe or primer may be obtained by biological synthesis (e.g. by digestion of a nucleic acid with a restriction endonuclease) or by chemical synthesis. For short sequences (up to about 100 nucleotides) chemical synthesis is desirable.

For longer sequences standard replication methods employed in molecular biology are useful, such as, for example, the use of M13 for single stranded DNA as described by Messing Methods Enzymol, 101: 20-78, 1983.

Other methods for oligonucleotide synthesis include, for example, phosphotriester and phosphodiester methods (Narang, editor, “Synthesis and Applications of DNA and RNA” Academic Press, New York (1987)) and synthesis on a support (Beaucage, et al., Tetrahedron Letters, 22: 1859-1862, 1981) as well as phosphoramidate technique, Caruthers, M. H., et al., “Methods in Enzymology,” Vol. 154, pp. 287-314 (1988), and others described in Narang (1987), and the references contained therein.

### Conjugates

In one example, a compound of the present disclosure is conjugated to an agent.

For example, the agent is selected from the group consisting of a radioisotope, a detectable label, a therapeutic compound, a colloid, a toxin, a nucleic acid, a peptide, a protein, an agent that increases the half-life of the compound in a subject and mixtures thereof.

The other compound can be directly or indirectly bound to the compound (e.g., can comprise a linker in the case of indirect binding). Examples of compounds include, a radioisotope (e.g., iodine-131, yttrium-90 or indium-111), a detectable label (e.g., a fluorophore or a fluorescent nanocrystal or quantum dot), a therapeutic compound (e.g., a chemotherapeutic or an anti-inflammatory), a colloid (e.g., gold), a toxin (e.g., ricin or tetanus toxoid), a nucleic acid, a peptide (e.g., a serum albumin binding peptide), a protein (e.g., a protein comprising an antigen binding domain of an antibody or serum albumin), an agent that increases the half-life of the compound in a subject (e.g., polyethylene glycol or other water soluble polymer having this activity) and mixtures thereof. Exemplary compounds that can be conjugated to a compound of the disclosure and methods for such conjugation are known in the art and described, for example, in WO2010/059821.

The compound may be conjugated to nanoparticles (for example as reviewed in Kogan et al., Nanomedicine (Lond). 2: 287-306, 2007). The nanoparticles may be metallic nanoparticles.

Some exemplary compounds that can be conjugated to a compound of the present disclosure are listed in Table 1.

TABLE 1
Compounds useful in conjugation.

| Group | Detail |
|---|---|
| Radioisotopes | ¹²³I, ¹²⁵I, ¹³⁰I, ¹³³I, ¹³⁵I, ⁴⁷Sc, ⁷²As, ⁷²Sc, ⁹⁰Y, ⁸⁸Y, |
| (either directly | ⁹⁷Ru, ¹⁰⁰Pd, ^{101m}Rh, ^{101m}Rh, ¹¹⁹Sb, ¹²⁸Ba, ¹⁹⁷Hg, ²¹¹At, |
| or indirectly) | ²¹²Bi, ¹⁵³Sm, ¹⁶⁹Eu, ²¹²Pb, ¹⁰⁹Pd, ¹¹¹In, ⁶⁷Gu, ⁶⁸Gu, |
| | ⁶⁷Cu, ⁷⁵Br, ⁷⁶Br, ⁷⁷Br, ^{99m}Tc, ¹¹C, ¹³N, ¹⁵O, ¹⁸I, ¹⁸⁸Rc, |
| | ²⁰³Pb, ⁶⁴Cu, ¹⁰⁵Rh, ¹⁹⁸Au, ¹⁹⁹Ag or ¹⁷⁷Lu |
| Half-life | Polyethylene glycol |
| extenders | Glycerol |
| | Glucose |
| Fluorescent | Phycoerythrin (PE) |
| probes | Allophycocyanin (APC) |
| | Alexa Fluor 488 |
| | Cy5.5 |
| Biologics | fluorescent proteins such as Renilla luciferase, GFP |
| | immune modulators or proteins, such as cytokines, |
| | e.g., an interferon |
| | toxins |
| | an immunoglobulin or antibody or antibody variable |
| | region |
| | half-life extenders such as albumin or antibody |
| | variable regions or peptides that bind to albumin |
| Chemothera- | Taxol |
| peutics | 5-FU |
| | Doxorubicin |
| | Idarubicin |

In one example, a compound of the disclosure is conjugated to a chemotherapy agent.

In one example, a compound of the disclosure is conjugated to a maytansinoid, e.g., DM1 or DM4.

In another example, a compound of the disclosure is conjugated to an auristatin, e.g., MMAE or MMAD.

Selection of Compounds that Specifically Bind to BTN2A1

Suitable methods for selecting a compound (e.g., an antibody or antigen binding fragment thereof) that specifically binds to BTN2A1, or an epitope thereof, are available to those skilled in the art.

For example, a screen may be conducted to identify compounds capable of binding to BTN2A1. Any compound that binds to BTN2A1 is then screened to identify those that do not substantially bind to a related protein, e.g., BTN2A2.

For example, a phage display library displaying antibody fragments is screened with BTN2A1 or a soluble form thereof to identify proteins that bind thereto. One or more proteins related to BTN2A1 to which the antibody fragment is not to be able to detectably bind are then used to remove cross-reactive proteins. A screening process for immunization of a non-human mammal can also be devised based on the foregoing as can a screening method for identifying other compounds described herein.

### Assaying Activity of Compound

Compounds of the disclosure are readily screened for biological activity, e.g., as described below.

### Binding Assays

One form of such an assay is an antigen binding assay, e.g., as described in Scopes (In: Protein purification: principles and practice, Third Edition, Springer Verlag, 1994). Such a method generally involves labeling the compound (e.g., an antibody or antigen binding fragment) and contacting it with immobilized antigen. Following washing to remove non-specific bound protein, the amount of label and, as a consequence, bound compound is detected. Of course, the compound can be immobilized and the antigen labeled. Panning-type assays, e.g., as described herein can also be used.

### Determining Neutralization

In some examples of the present disclosure, a compound of the present disclosure neutralizes BTN2A1 activity.

Various assays are known in the art for assessing the ability of a compound to neutralize signaling of a protein.

In one example, the neutralization of BTN2A1 is determined by contacting melanoma cells with the compound such that the compound binds to the BTN2A1 forming a cell-compound complex; contacting the complex with a T cell (e.g., a CD4⁺T cell or a CD8⁺ T cell); and determining the level of death of the melanoma cells (e.g., cytotoxic killing of the melanoma cells by the T cells), wherein an increase in the level of death of the melanoma cells in the presence of the compound compared to in the absence of the compound indicates that the compound neutralized BTN2A1. For example, the melanoma cell and the T cells are HLA matched and the T cells recognize an antigen recognized by the melanoma cell (e.g., NY-ESO-1 or Melan A). In one example, a plurality of melanoma cells (e.g., about 2.5×10⁴ cells) are contacted with a plurality of T cells (e.g., about 12.5×10⁴ T cells). Loss of viability of melanoma cells is then assessed, e.g., by determining loss of viability, e.g., using an assay to assess MTS reduction to formazan salt (Promega Corporation).

In one example, the neutralization of BTN2A1 is determined by contacting a melanoma cell with the compound such that the compound binds to the BTN2A1 forming a cell-compound complex; contacting the complex with T cells (e.g., CD4⁺ T cells or CD8⁺ T cells); and determining the level of activation of the T cells (e.g., by determining the level of interferon (IFN) γ or tumor necrosis factor (TNF) α) production, wherein an increase in the level of activation of the T cells in the presence of the compound compared to in the absence of the compound indicates that the compound neutralized BTN2A1. For example, the melanoma cell and the T cells are HLA matched and the T cells recognize an antigen recognized by the melanoma cell (e.g., NY-ESO-1 or Melan A). In one example, a plurality of melanoma cells (e.g., about 10⁵ cells) are contacted with a plurality of T cells (e.g., about 2.5×10⁴ T cells). In one example, activation is determined by fluorescence activated cell sorting in which cells are stained for cell surface CD3 and CD8 and IFNγ and/or TNFα production. The number of CD8⁺ IFNγ⁺ and/or CD8⁺ TNFα⁺ cells are then determined.

An ELISA or ELISPOT assay can alternatively be used to assess the amount of cytokine secreted by T cells.

Other methods for assessing neutralization of BTN2A1 signaling are contemplated by the present disclosure.

### Determining Effector Function

Methods for assessing ADCC activity are known in the art.

In one example, the level of ADCC activity is assessed using a ⁵¹Cr release assay, a europium release assay or a ³⁵S release assay. In each of these assays, cells expressing BTN2A1 are cultured with one or more of the recited compounds for a time and under conditions sufficient for the compound to be taken up by the cell. In the case of a ³⁵S release assay, cells expressing BTN2A1 can be cultured with 35S-labeled methionine and/or cysteine for a time sufficient for the labeled amino acids to be incorporated into newly synthesized proteins. Cells are then cultured in the presence or absence of a compound of the disclosure and in the presence of immune effector cells, e.g., peripheral blood mononuclear cells (PBMC) and/or NK cells. The amount of ⁵¹Cr, europium and/or ³⁵S in cell culture medium is then detected, and an increase in the presence of the compound compared to in the absence of the compound indicates that the antibody or antigen binding fragment has effector function. Exemplary publications disclosing assays for assessing the level of ADCC induced by a compound include Hellstrom, et al. Proc. Natl Acad. Sci. USA 83:7059-7063, 1986 and Bruggemann, et al., J. Exp. Med. 166:1351-1361, 1987.

Other assays for assessing the level of ADCC induced by a compound include ACTI™ nonradioactive cytotoxicity assay for flow cytometry (CellTechnology, Inc. CA, USA) or CytoTox 96® non-radioactive cytotoxicity assay (Promega, WI, USA).

Alternatively, or additionally, effector function of a compound is assessed by determining its affinity for one or more FcγRs, e.g., as described in U.S. Pat. No. 7,317,091.

C1q binding assays may also be carried out to confirm that the compound is able to bind C1q and may induce CDC. To assess complement activation, a CDC assay may be performed (see, for example, Gazzano-Santoro et al, J. Immunol. Methods 202: 163, 1996.

### Determining Affinity

Optionally, the dissociation constant (Kd) or association constant (Ka) or equilibrium constant (K_{D}) of a compound for BTN2A1 or an epitope thereof is determined. These constants for a compound (e.g., an antibody or antigen binding fragment) are, in one example, measured by a radiolabeled or fluorescently-labeled BTN2A1-binding assay. This assay equilibrates the compound with a minimal concentration of labeled BTN2A1 (or a soluble form thereof, e.g., comprising an extracellular region of BTN2A1 fused to an Fc region) in the presence of a titration series of unlabeled BTN2A1. Following washing to remove unbound BTN2A1, the amount of label is determined.

Affinity measurements can be determined by standard methodology for antibody reactions, for example, immunoassays, surface plasmon resonance (SPR) (Rich and Myszka Curr. Opin. Biotechnol 11:54, 2000; Englebienne Analyst 123: 1599, 1998), isothermal titration calorimetry (ITC) or other kinetic interaction assays known in the art.

In one example, the constants are measured by using surface plasmon resonance assays, e.g., using BIAcore surface plasmon resonance (BIAcore, Inc., Piscataway, N.J.) with immobilized BTN2A1 or a region thereof. Exemplary SPR methods are described in U.S. Pat. No. 7,229,619.

### Assessing Therapeutic Efficacy

Various in vitro assays are available to assess the ability of a compound of the disclosure to treat a disease or condition described herein. Exemplary assays for determining activation of T cells or cytotoxicity of T cells are described above as are methods for determining ADCC and CDC. These assays are readily adapted to determining the ability of a cell to kill a melanoma cells (e.g., by determining cell proliferation levels in the presence of a compound).

In one example, the efficacy of a compound to treat a disease or condition is assessed using an in vivo assay.

In one example, a xenotransplantation model of a cancer, e.g., melanoma is used to assess therapeutic efficacy. For example, mice (e.g., NOD/SCID mice) are administered cancer cells, e.g., melanoma cells and a compound of the disclosure is administered to the mice and the level of size of any tumor or the presence of any tumor or metastases thereof is assessed. A reduction in the size or number of tumors or metastases in the presence of the compound compared to in the absence of the compound indicates therapeutic efficacy.

### Compositions

Suitably, in compositions or methods for administration of the compound of the disclosure to a mammal, the compound is combined with a pharmaceutically acceptable carrier as is understood in the art. Accordingly, one example of the present disclosure provides a composition (e.g., a pharmaceutical composition) comprising the compound of the disclosure combined with a pharmaceutically acceptable carrier. In another example, the disclosure provides a kit comprising a pharmaceutically acceptable carrier suitable for combining or mixing with the compound prior to administration to the mammal. In this example, the kit may further comprise instructions for use.

In general terms, by “carrier” is meant a solid or liquid filler, binder, diluent, encapsulating substance, emulsifier, wetting agent, solvent, suspending agent, coating or lubricant that may be safely administered to any mammal, e.g., a human. Depending upon the particular route of administration, a variety of acceptable carriers, known in the art may be used, as for example described in Remington's Pharmaceutical Sciences (Mack Publishing Co. N.J. USA, 1991).

By way of example only, the carriers may be selected from a group including sugars (e.g. sucrose, maltose, trehalose, glucose), starches, cellulose and its derivatives, malt, gelatine, talc, calcium sulfate, oils inclusive of vegetable oils, synthetic oils and synthetic mono- or di-glycerides, lower alcohols, polyols, alginic acid, phosphate buffered solutions, lubricants such as sodium or magnesium stearate, isotonic saline and pyrogen-free water. For example, the carrier is compatible with, or suitable for, parenteral administration. Parenteral administration includes any route of administration that is not through the alimentary canal. Non-limiting examples of parenteral administration include injection, infusion and the like. By way of example, administration by injection includes intravenous, intra-arterial, intramuscular and subcutaneous injection. Also contemplated is delivery by a depot or slow-release formulation which may be delivered intradermally, intramuscularly and subcutaneously.

### Combination Therapy

The compounds of the disclosure and/or of the methods of the disclosure can be used either alone or in combination with other agents in a therapy for the treatment of cancer, e.g., melanoma.

For instance, a compound of the disclosure or of use in a method of the disclosure is co-administered with at least one additional therapeutic agent. For example, the additional therapeutic agent is a chemotherapeutic agent.

In one example, the chemotherapy agent is, for example, caboplatin, cisplatin, cyclophosphamide, docetaxal, doxorubicin, erlotinib, etoposide, fluorouracil, irinotecan, methotrexate, paclitaxel, topotecan, vincristine or vinblastine. In one example, the chemotherapy agent is selected from the group consisting of methotrexate, 1-asparaginase, vincristine, doxorubicin, danorubicin, cytarabine, idarubicin, mitoxantrone, cyclophosphamide, fludarabine, chlorambucil and combinations thereof.

In some examples, the additional therapeutic agent may be Aldesleukin, Dacarbazine, DTIC-Dome (Dacarbazine), Ipilimumab, Proleukin (Aldesleukin), Vemurafenib, Yervoy (Ipilimumab), and/or Zelboraf (Vemurafenib).

In some examples, the additional therapeutic agent is an immunotherapy, e.g., that neutralizes a regulator of an immune response (e.g., CTLA-4 or PD-1 or PD-L1) such as Yervoy (Ipilimumab), nivolumab, lambrolizumab or MPDL-3280A.

In some examples, compounds of the disclosure are used for the treatment of cancer, e.g., melanoma in an individual in combination with radiation therapy. In some examples, compounds of the disclosure are used for the treatment of cancer, e.g., melanoma in an individual in combination with surgical removal of all or a portion of the cancer, e.g., melanoma from the individual.

In some examples of the disclosure, the subject has been previously treated for cancer, e.g., melanoma, for example, using an anti-cancer therapy. In one example, the anti-cancer therapy is surgery. In another example, the subject can be further treated with an additional anti-cancer therapy before, during (e.g., simultaneously), or after administration of the compound of the disclosure. Examples of anti-cancer therapies include, without limitation, surgery, radiation therapy (radiotherapy), biotherapy, immunotherapy, chemotherapy, or a combination of these therapies

### Dosages and Timing of Administration

For the treatment of a disease or condition, the appropriate dosage of a compound active agent (e.g., an antibody or antigen binding fragment of the disclosure), will depend on the type of disease to be treated, the severity and course of the disease, whether the compound is administered for preventive or therapeutic purposes, previous therapy, the patient's clinical history and response to the compound, and the discretion of the attending physician. The particular dosage regimen, i.e., dose, timing, and repetition, will depend on the particular individual and that individual's medical history as assessed by a physician. Typically, a clinician will administer a compound until a dosage is reached that achieves the desired result.

Methods of the present disclosure are useful for treating, ameliorating or preventing the symptoms of diseases or conditions in a mammal, or for improving the prognosis of a mammal. Methods of the present disclosure are also useful for delaying development of or preventing diseases or condition in an individual at risk of developing the disease or condition or a relapse thereof.

For administration of the compounds described herein, normal dosage amounts may vary from about 10 ng/kg up to about 100 mg/kg of an individual's body weight or more per day. For repeated administrations over several days or longer, depending on the severity of the disease or disorder to be treated, the treatment can be sustained until a desired suppression of symptoms is achieved.

In some examples, the compound (e.g., a polypeptide based compound, such as an antibody or antigen binding fragment) is administered at an initial (or loading) dose of between about 1 mg/kg to about 30 mg/kg. The compound can then be administered at a maintenance dose of between about 0.0001 mg/kg to about 10 mg/kg. The maintenance doses may be administered every 7-30 days, such as, every 10-15 days, for example, every 10 or 11 or 12 or 13 or 14 or 15 days.

In the case of a mammal that is not adequately responding to treatment, multiple doses in a week may be administered. Alternatively, or in addition, increasing doses may be administered.

In another example, for mammals experiencing an adverse reaction, the initial (or loading) dose may be split over numerous days in one week or over numerous consecutive days.

Dosages for a particular compound may be determined empirically in mammals that have been given one or more administrations of the antibody or antigen binding fragment. To assess efficacy of a compound, a clinical symptom of a disease or condition can be monitored.

Administration of a compound according to the methods of the present disclosure can be continuous or intermittent, depending, for example, on the recipient's physiological condition, whether the purpose of the administration is therapeutic or prophylactic, and other factors known to skilled practitioners. The administration of a compound may be essentially continuous over a preselected period of time or may be in a series of spaced doses, e.g., either during or after development of a condition.

The present disclosure includes the following non-limiting examples.

### Example 1: Materials and Methods

A Fab, Hu34C1, was produced and shown to specifically bind to melanoma cells and not substantially to peripheral blood mononuclear cells. This antibody was reformatted into an intacg IgG format and cloned into a mammalian expression vector.

### Cell Culture

FreeStyle 293 cells were cultured in FreeStyle Expression Medium supplemented with penicillin/streptomycin/fungizone reagent. Prior to transfection the cells were maintained at 37° C. in humidified incubators with 8% CO₂.

### Transient Transfection

Transient transfection of FreeStyle 293 cells with the mammalian expression vectors was performed using 293fectin transfection reagent according to the manufacturer's instructions. Cells (30 ml) were transfected at a final concentration of 1×10⁶ viable cells/ml and incubated with shaking in a non-baffled 125 mL flask (Corning) for 5 days at 37° C. with 8% CO2. 4 hours post-transfection the cell cultures were supplemented with Lupin to a final concentration of 0.5% v/v. The cell culture supernatants were harvested by centrifugation at 2500 rpm and were then passed through a 0.22 μM filter prior to purification.

### Antibody Purification

Monoclonal antibodies were purified using tandem protein A affinity chromatography and desalting column chromatography. Chromatography using Hitrap MabSelect SuRe and HiPrep 26/10 desalting resins was performed using an AKTA express (GE Healthcare, UK) as per manufacturer's recommended method. Briefly, equilibration of the Protein A affinity column was performed in 1×MT-PBS buffer. The filtered conditioned cell culture media (25 ml) was applied to the column at 1 ml/min and washed sequentially with 1×MT-PBS (10 ml) and 10 mM Tris, 0.5M Arginine, 150 mM NaCl 150 mM NaCl pH 7.2 (6 ml). The bound antibody was then eluted with 0.1M Na Acetate pH 3.0 (8 ml) and subsequently applied to the desalting column. The antibody concentration was determined using absorbance at 280 nm. Protein fractions were pooled and concentrated using an Amicon UltraCel 50K centrifugal device prior to sterile filtration using 0.22 um filters.

The purity of the antibody was analyzed by SDS-PAGE, where 2 μg protein in reducing Sample Buffer was loaded onto a Novex NuPAGE 4-12% Bis-Tris Gel and a constant voltage of 200V was applied for 40 minutes in an XCell SureLock Mini-Cell (Invitrogen, CA) with NuPAGE MES SDS running buffer before being visualized using Coomassie Stain, as per the manufacturer's instructions.

### Identification of BTN2A1 as the Melanoma Antigen Recognized by Antibody Hu34C1

### Generation of Covalently Coupled Antibody Resin

Antibody Hu34C1-G4Fabk-mG2aFC-RF11-82 was covalently coupled at 1.8 mg/ml to NHS activated agarose (Pierce NHS activated Agarose slurry 26200). Coupling efficiency of greater than 90% was observed. Control resin with an unrelated Isotype matched control mAb (BM4) was also generated.

### Generation of Cellular Lysate

The adherent melanoma cell line LM-Mel-62 was cultured in RPMI 1640 media (Sigma #R0883) supplemented with 10% FCS (Sigma #12003), 50U/ml Penicillin and 50 μg/ml Streptomycin (Pen-Strep, Gibco #15070-063), 2 mM Glutamax (Gibco #35050) at 37° C., 5% CO₂. To generate whole cell lysate, culture media was removed, the cells washed 1× with PBS and 10 ml of fresh ice cold PBS per 15 cm plate added for 30 minutes at 4° C. The cells were then removed by pipetting and cell scraping where necessary, pelleted by centrifugation at 250 g, 5 minutes, 4° C. and lysed in an NP40 based lysis buffer (0.025 M Tris, pH 7.5, 0.15 M NaCl, 0.001M EDTA, 1% NP40, 5% glycerol, freshly supplemented with protease inhibitor cocktail—Roche Complete Protease inhibitor). After 1 hr at 4° C. on a rotating wheel, nuclear and cellular debris was removed by centrifugation (13,000 g, 10 minutes, 4° C., Eppendorf centrifuge) and the lysate frozen until required. Immediately prior to use, lysate was thawed, re-centrifuged (4000 g, 10 minutes) to remove any precipitated material and filtered through a 0.2 micron filter.

### Hu34C1 Antigen Immunoprecipitation

For the identification of the Hu34C1 antigen, cellular lysate from 80×15 cm confluent plates of LM-Mel-62 cells (approximately 50 ml lysate) was used. The lysate was first pre-cleared extensively by passing multiple times through 6 ml of control antibody coupled resin. The pre-cleared material was then incubated with 0.5 ml of Antibody 34C1 covalently coupled beads on a rotating wheel at 4° C. After 3 hr, the mix was gradually transferred to a 10 ml BioRad column and the collected lysate passed through the captured antibody resin twice. The antibody resin was then extensively washed with ice-cold NP40 lysis buffer (30 mls) and ice-cold PBS (20 mls) before elution with an acidic elution buffer (IgG Immunopure elution buffer-Pierce). The eluate was concentrated using an Amicon centrifugation device (10 kDa cut-off). A small amount of concentrate was kept for direct analysis by mass spectrometry and the rest supplemented with Reducing SDS PAGE sample buffer, incubated at 75° C. for 5 minutes, and proteins separated on a 4-12% Tris-Bis Novex gel. Proteins were initially visualized by staining with Sypro Ruby (Molecular Probes) and then Phast Blue Coomassie (Pharmacia) prior to the excision of protein bands for Mass spectrometric identification.

### Mass Spectrometry

Gel fragments were washed with 100 mM ammonium bicarbonate (AmBiC) prior to reduction. Bands were reduced with 10 mM DTT for 1 hr at room temperature, immediately followed by alkylation with 55 mM Iodoacetamide for 1 hr at room temperate in the dark. Excess DTT and Iodoacetamide were removed via washing with 100 mM AmBiC and acetonitrile (MeCN). Gel bands were dehydrated in a vacuum centrifuge (EZ-2 plus, GeneVac, New York, USA) for 20 mins. The dry gel pieces were rehydrated using a solution of 100 mM AmBiC containing 12.5 ng μL⁻¹ Trypsin. The gel bands were incubated at 4° C. for 45 mins. Excess supernatant was removed and replaced with 100 mM AmBiC. The sample was digested overnight at room temperature. Peptides were extracted from the gel bands via sequential dehydration (50% (v/v) MeCN, 1% (v/v) formic acid) and rehydration (50 mM AmBiC). The solution collected at each stage was pooled and reduced to 1-2 μL in a vacuum centrifuge (EZ-2 plus, GeneVac, New York, USA). The sample was reconstituted with 0.1 M acetic acid to a final volume of 20 μL. Each gel band sample was subsequently analyzed on an AB Sciex 5600 Triple TOF (AB Sciex, Mulgrave, Australia) equipped with an Eksigent NanoUltra cHiPLC system (AB Sciex, Mulgrave, Australia). The peptides were separated on a linear gradient from 0-30% MeCN over 33 mins (300 nL min⁻¹). The instrument was run in IDA mode targeting the top 20 precursor ions. Data was searched using the MSPF MSILE pipeline using the MASCOT search engine. Briefly, the search parameters were set as: Enzyme=Trypsin, Fixed Modifications=Carbamidomethyl (Cys), Variable Modifications=Oxidation (Met), 50 ppm tolerance on peptide mass, 0.3 Da tolerance on fragment mass, 2 missed cleavages allowed, Instrument type=ESI-Q-TOF, Swissprot database limited to Human sequences was selected. All search results were manually validated.

### BTN2A1 Antigen Validation by ELISA

For ELISA, Nunc Maxisorp immunoplates were coated overnight at 4° C. with 100 μL/well of 1 μg/mL of the appropriate antigen in PBS. Negative control wells coated with PBS alone were also included. Wells were then blocked for 2 hrs at 37° C. with 200 μL of 5% skim milk/PBS, and washed 3× in PBST. Appropriate antibodies were added to a final concentration of 10 μg/mL in TBS-T containing 2% skim milk powder and plates were incubated at room temperature for 60 min. The wells were washed 5 times with TBS-T and incubated with HRP-conjugated anti-human IgG antibody. Following incubation for 60 min at RT, the wells were washed as before and bound antibodies were detected using 100 μL/well TMB/E substrate. The wells were incubated at RT for 10 min after which the reaction was terminated by addition of 0.5 volumes of 2 M H₃PO₄ and the resulting signal was measured by absorbance at 450 nm in a microplate reader.

### Cell Culture

Melanoma cell lines were established from fresh patient tumors by mechanical dissociation of tissue with subsequent overnight digestion in media containing collagenase IV at 37° C. All cancer cell lines used were grown in RPMI1640 (LifeTechnologies, Carlsbad, Calif. 92010, USA) supplemented with 10% FCS. Fibroblasts and melanocytes were purchased from Lonza and grown in their respective special growth media (All Lonza, Lonza Inc., NJ 07401, USA).

### Immuno-Fluorescence

LM-Mel-62 was plated out in 12 well plates at 40,000 cells per well. The next day cells were washed, fixed with 4% PFA for 10 min and stained using 20 μg/ml BTN2A1 ab in 500 ml PBS for 10h at 4° C. The next day, cells were washed twice in PBS, and the secondary anti-mouse PE antibody added at a 1:100 dilution for 1h at room temperature followed by 2 washing steps with PBS. DAPI (LifeTechnologies) was added at a final concentration of 300 nM as per manufacturer's protocol. Pictures were taken with a Olympus microscope and the appropriate filters at 20× magnification.

### Flow-Cytometry

Cells were trypsinized, washed twice with PBS. Cells were stained with the BTN2A1 antibody at 10 μg/ml in PBS for 30 min at 4° C. in the dark. Cells were washed twice with PBS and incubated for 20 min 4° C. in the dark with an anti-mouse IgG-PE labeled secondary antibody (Jackson-Immuno Research Laboratories, PA, USA 19390) at a 1:100 dilution in PBS. Following two wash steps with PBS, cells were stained with a LIVE/DEAD fixable dead cell stain (LifeTechnologies) according to the manufacturer's protocol. Cells were filtered through a cell strainer and run on a FacsCantoII (Becton, Dickinson and Company) with appropriate laser settings. Gates were set on live cells only and binding compared to isotype. All data analysis was performed using FlowJo (TreeStar, Ashland, Oreg. 97520 USA) and statistical analysis where appropriate was performed using PRISM (GraphPad Software, Inc, La Jolla, Calif. 92037 USA) software.

### Immuno Histochemistry on Melanoma Cell Lines and Melanoma Tissues

Cell blocks were prepared by growing the melanoma cell lines to 80% confluency and a cell pellet was collected. 150 μl of normal human serum was added and the cell pellet detached from the bottom of the tube with a wooden applicator stick. 150 μl thrombin (Warner Lambert Company, New Jersey 07950, USA) was added and mixed. The forming clot was transferred to Tally Ho paper and placed in a histology cassette in 10% formalin for standard embedding in paraffin. Human tissue specimens were collected from patients and fixed in 10% neutral buffered formalin for standard embedding in paraffin.

Cell/tissue blocks were sectioned on a microtome at the thickness of 4 μM. Sections were put onto charged glass slides and allowed to dry overnight at 37° C. Section/slides were de-waxed in 100% Xylene and rehydrated in 100% ethanol followed by 5 mins in 70% ethanol, and 1×5 mins in tap water. Slides were then washed in PBST (3×5 mins), before quenching endogenous peroxidase activity by incubating in 3% hydrogen peroxide (diluted in dH2O) for 15 mins at RT. Slides were then washed in PBST (3×5 mins), before antigen retrieving in citrate buffer [pH 6] (Thermo Scientific) in a microwave pressure cooker for 20 mins on HIGH followed by 3 washes in PBS before blocking non-specific staining with SuperBlock® (Thermo Fisher Scientific, MA 02454, USA) for 45 mins at RT. Primary antibodies diluted in Dako Antibody Diluent (Dako Denmark A/S, DK-2600, Denmark) to 3 μg/mL. Sections were incubated with primary antibodies (Mouse anti BTN2A1 MAb: CSL 34C1-G4FabK-mG2aFc-aMIC or IgG2a Isotype Control: CSL BM4-95F-1B7) overnight at 4° C. Slides were washed in PBS before incubating with anti-mouse EnVision+System HRP secondary antibody for 45 mins at RT. Secondary antibody used neat (Dako) and slides were again washed in PBS before developing stain with DAB Chromogen (Dako) for ˜90 seconds. Slides counterstained with haematoxylin and Scott's Water, before dehydrating in 70% ethanol (10 mins); followed by 5 mins in 100% ethanol. Slides then incubated in 100% Xylene, before mounting coverslips.

qPCR for BTN2A1 Expression

BTN2A1 primers were designed using Primer3. RNA was extracted from cell lines or tumors using the RNEasy kit (Qiagen) according to the manufacturer's protocol and reverse transcribed using the HighCapacity Reverse transcription Kit (LifeTechnologies) according to the manufacturer's instructions. qPCR was performed using a VII7 qPCR machine (LifeTechnologies) and SensiFast master mix (Bioline, London NW2 6EW, UK). Copy numbers were calculated per 10,000 Beta-Actin (house keeping gene) copies.

### Nanostring Analysis of Melanoma Samples

Cores from 12 metastatic melanoma samples (paraffin-embedded) were used for RNA extraction using the High Pure FFPE RNA Micro Kit from Roche. Briefly, paraffin was removed with 100% Xylene, cell pellet washed with 70% ethanol and dried at 55 degrees C. Pellet was incubated with Tissue Lysis Buffer and Proteinase K at 55 degrees C. for 4 hrs followed by RNA extraction using the supplied filter tubes. Concentration of RNA was quantified by NanoDrop and quality controlled using a Bioanalyser.

250 μg RNA were used for the subsequent NanoString assay as per manufacturers recommendations (NanoString Technologies, Inc. Seattle, Wash. 98109, USA). Analysis was performed on a nCounter Analysis System at the Peter MacCallum Cancer Centre, Melbourne, Australia). Data are expressed as absolute counts.

siRNA Mediated Knockdown of BTN2A1

Two BTN2A1 targeting siRNAs or a negative control siRNA (LifeTechnologies) were transfected into melanoma cells at 10 nM using RNAiMaxx (LifeTechnologies) as per manufacturer's protocol. After 24h media was changed to complete growth media and knockdown efficiency determined by qPCR and/or flow-cytometry 48h after transfection.

### Proliferation Assay.

PBMC were purified from healthy donor buffy coats (Red Cross, Melbourne) by density gradient centrifugation over Ficoll-Hypaque. Cells were resuspended at 10⁶/ml in PBS/0.1% BSA and CFSE (Invitrogen) was added to 1 μM final concentration. Cells were incubated at 37° C., 5% CO2 for 10 min, then a 5× volume of ice-cold TCRPMI media (containing RPMI, 10% Human serum, Penicillin/Streptomycin, Glutamate, non-essential amino acids, sodium pyruvate) was added and cells incubated for 5 min on ice. Cells were washed twice in TCRPMI. Wells of a NUNC MaxiSorb96 well plate (eBioscience, Inc. San Diego, Calif. 92121, USA) were coated overnight at 4° C. with 10 μg/ml or 1 μg/ml recombinant BTN2A1 protein (Origene, Rockville, Md. 20850, USA) in PBS, or BSA as a control. Plates were rinsed once with PBS. 2×10⁵ PBMC were plated per well. To each condition (i.e. BTN2A1 or BSA coated wells) either antagonistic anti-CD3 antibody OKT3 (ebioscience) (10 μg/ml final concentration) or anti-CD3/CD28 dynabeads (LifeTechnologies) at a cell:bead ratio of 1:1 were added. Equivalent volumes of media were added to control wells. Cells were incubated at 37° C. for 72 h. Cells were stained with fluorescent antibodies for CD3, CD4 and CD8, and samples were run on a FACS Canto. FlowJo software was used for analysis, including determination of the percentage of proliferating CD4⁺ or CD8⁺ cells based on dilution of CFSE.

### Induction of Regulatory T Cells.

PBMC were purified from healthy donor buffy coats (Red Cross, Melbourne) by density gradient centrifugation over Ficoll-Hypaque. A portion of PBMC was resuspended at 80 μl/10⁷ cells in PBS/0.5% BSA/2 mM EDTA. Magnetic anti-CD4 beads (Miltenyi biotech) were added at 20 μl/10⁷ cells. Cells were incubated at 4° C. for 15 min, and then washed. CD4⁺ cells were isolated by separation using an AutoMACS (Miltenyi biotech) and program ‘Possel’. Wells of a 96 well plate were coated overnight at 4° C. with 10 μg/ml or 1 μg/ml recombinant BTN2A1 protein in PBS, or BSA as a control. Plates were rinsed once with PBS. Either PBMC or sorted CD4+ cells were plated at 2×10⁵ per well and incubated at 37° C., 5% CO2 for 72 h. Cells were surface stained with fluorescent antibodies for CD3, CD4 and CD25, then stained intracellularly for FoxP3 using the FoxP3 intracellular staining kit (ebioscience). Samples were run on a FACS Canto and analyzed using FlowJo software. The percentage of CD4⁺CD25⁺ FoxP3⁺ regulatory T cells was assessed for each condition.

### Gene-Expression Analysis

RNA was analyzed on Illumina HT-12 v3 arrays at the Australia Genome Research Facility (AGRF, Australia). Raw data were read in to the R environment for statistical computing (http://www.rproject.org/) using the limma package, background was corrected using the normexp function, and log 2 transformed and quantile normalized.

### T Cell Activation Assay

Melanoma cell lines which were HLA-matched and positive for expression of NY-ESO-1, or Melan A were selected from our cell line database. Cells were plated out in 12 well plates and were transfected with either scrambled, or two different BTN2A1 specific siRNAs as described above and incubated at 37° C., 5% CO2 for 48 h. Specific BTN2A1 knockdown at this timepoint was confirmed by RT-PCR. Melanoma cells were plated in a 96 well plate at 10⁵ cells/well. T cell clones recognizing either NY-ESO-1 HLA-Cw*0304/*0303 restricted epitopes 96-104 and 124-133, or Melan A HLA-A*0201 epitope 25-36 were added at 2.5×10⁴ per well in TCRPMI and Brefeldin A. Cells were incubated at 37° C., 5% CO2 for 4 h. Cells were surface stained with fluorescent antibodies for CD3 and CD8, then stained intracellularly for IFNγ and TNFα using fixation and permeablization reagents from BD biosciences. Samples were run on a FACS Canto and analyzed using FlowJo software. The percentage of CD8⁺ IFNγ⁺or CD8⁺ TNFα⁺ activated T cells was determined for each condition.

### Cytotoxicity Assays.

Melanoma cell lines which were HLA-matched and positive for expression of NY-ESO-1, or Melan A were selected from our cell line database. Cells were transfected with either scrambled, or two different BTN2A1 specific siRNAs as described above and incubated at 37° C., 5% CO2 for 48 h. Specific BTN2A1 knockdown at this timepoint was confirmed by RT-PCR. Melanoma cells were plated in a 96 well plate at 2.5×10⁴ cells/well. T cell clones recognizing either NY-ESO-1 HLACw*0304/*0303 restricted epitopes 96-104 and 124-133, or MelanA HLA-A*0201 epitope 25-36 were added at 12.5×10⁴ per well (5:1 effector to target ratio) in duplicate. Cells were incubated at 37° C., 5% CO2 for 16-20 h. The cytotoxicity of T cells to melanoma cells was determined using an MTS assay (CellTiter 96 Aqueous One Solution Cell Proliferation Assay, Promega, WI 53711-5399, USA).

ADCC Assays with Hu34C1.

BTN2A1-positive target cells (either LM-Mel-62 or 293FS) (about 1×10⁴ cells) were incubated with anti-BTN2A1 antibody (34C1) and NK cells at an effector to target cell ratio (E:T) of 10:1 at 37° C. in RPMI/5% FCS for 18h. Lactate dehydrogenase (LDH) release was measured using the CytoTox-One reagent (Promega). Specific lysis was determined by normalizing the data to maximal (detergent) and background (effector and target cells alone) lysis.

PBMCs from healthy donors were incubated with BTN2A1-positive target cells (LM-Mel-62) (Behren et al., Pigment Cell and Melanoma Research, DOI: 10.1111/pcmr.12097, 2013) in the presence and absence of anti-BTN2A1 antibody (34C1) for 4h in RPMI/10% FCS at 37° C. NK cell activation was determined by examining the proportion of NK cells (CD3⁻, CD56⁺) that were CD107a⁺by flow cytometry as previously described (Penack et al. Leukemia. 19: 835-840, 2005). The mean and SD from 4 experiments is shown.

### Example 2: Results

### Identification of BTN2A1 as Antigen Recognized by mAb Hu34C1

To identify the melanoma antigen recognized by mAb Hu34C1, large scale immunoprecipitation was conducted using covalently coupled antibody beads and total cellular extract from the melanoma cell line LM-Mel-62 as described in the methods section. A prominent protein band was detected in the eluted protein immunoprecipitate, not evident in a control immunoprecipitate with a different melanoma specific antibody. The protein band was excised for mass spectrometric identification. In addition, some of the direct eluate was also analyzed.

MS/MS analysis revealed the major band to be BTN2A1, with extensive coverage in peptides identified. The identity of BTN2A1 as the antigen for mAb Hu34C1 was further consolidated by an ELISA conducted with commercially obtained hu sBTN2A1.

### BTN2A1 Expression on Different Melanoma Cell Lines

The expression of BTN2A1 and other family members of the butyrophilin superfamily was then assessed in early passage melanoma cell lines. Expression of BTN2A1 was detected in the cells. Other than BTN3A2 none of the other family members were highly expressed as detected by Illumina HT12 v3 gene-expression arrays.

The BTN2A1 antibody was tested on LM-Mel-62 cells and shown to bind to the surface of living melanoma cells in a highly specific manner. This was true for all melanoma cell lines tested.

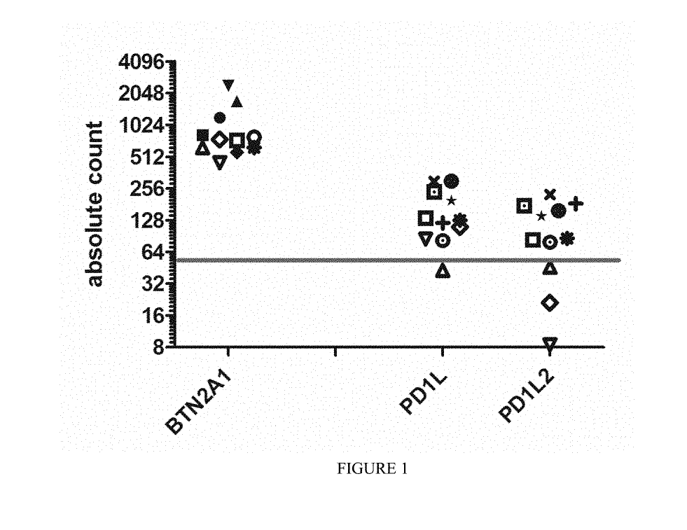

Nanostring analysis of melanoma samples also showed high expression of BTN2A1. This level was consistently higher than PD1L1 and PD1L2, current targets for melanoma therapy (FIG. 1); and all melanoma samples tested were positive (above the cut-off value) for BTN2A1 transcripts.

### BTN2A1 Expression on Other Cancer Cell Lines and Normal Cells

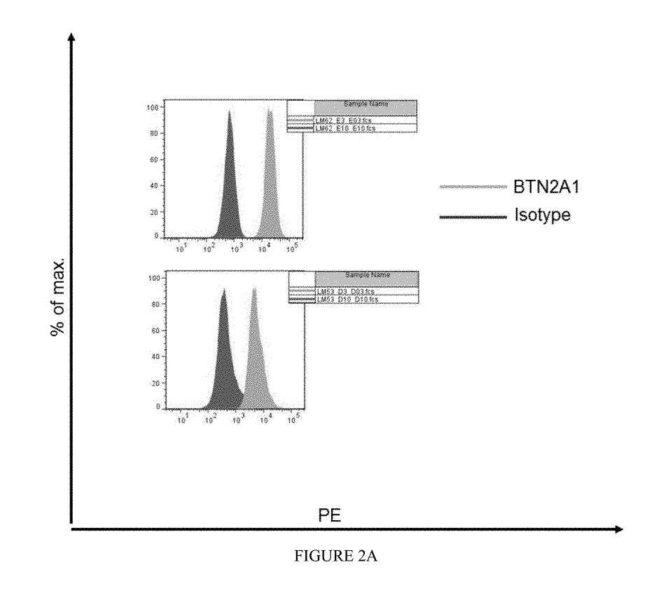

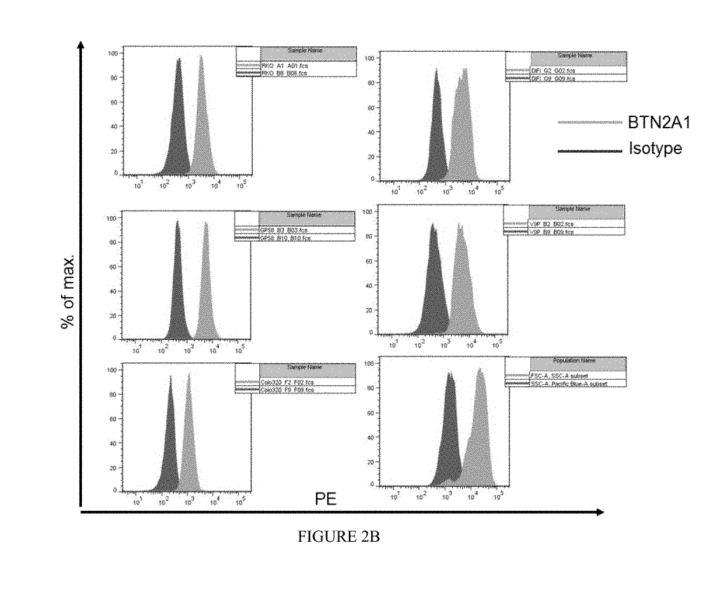

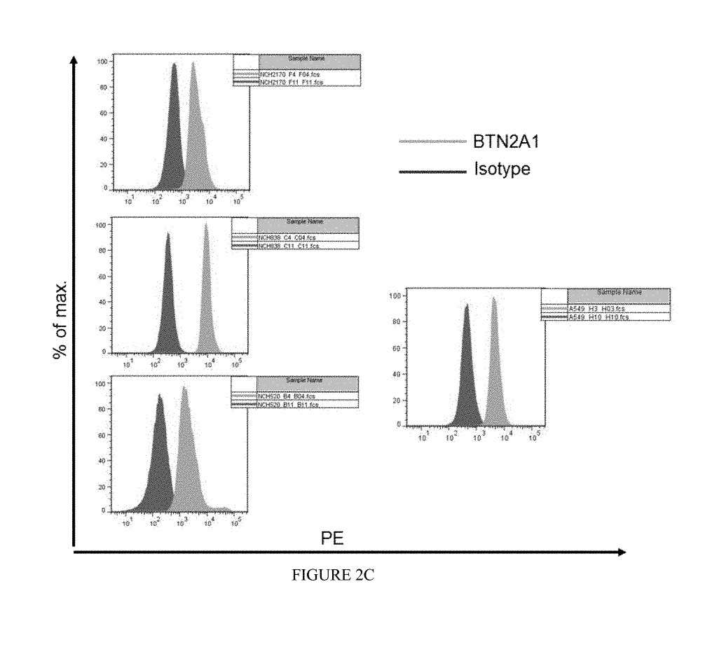

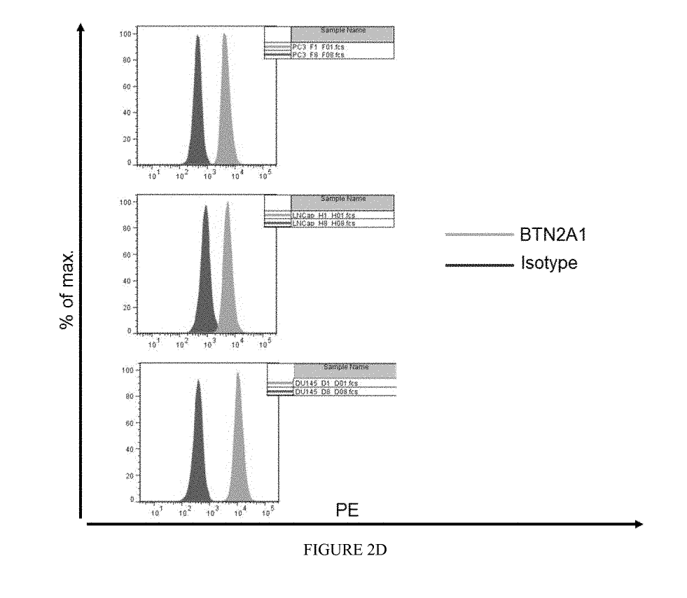

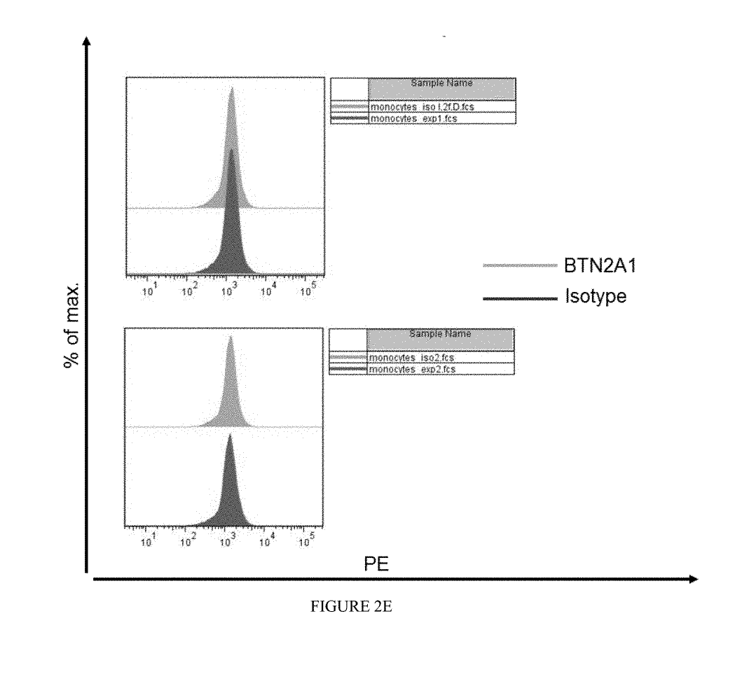

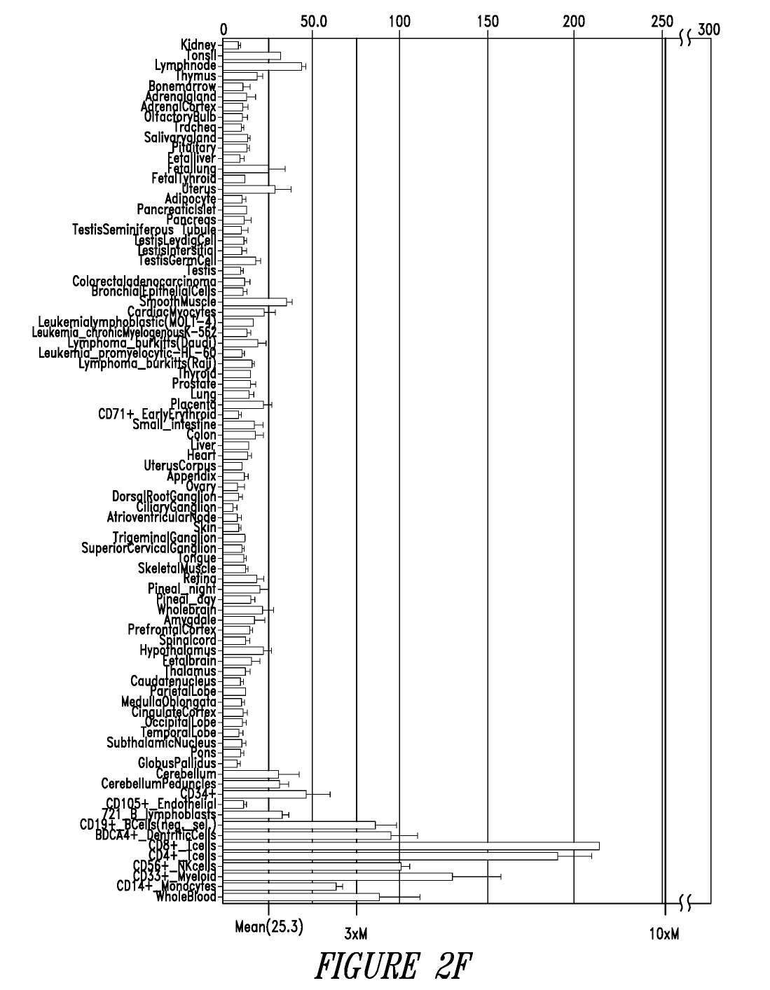

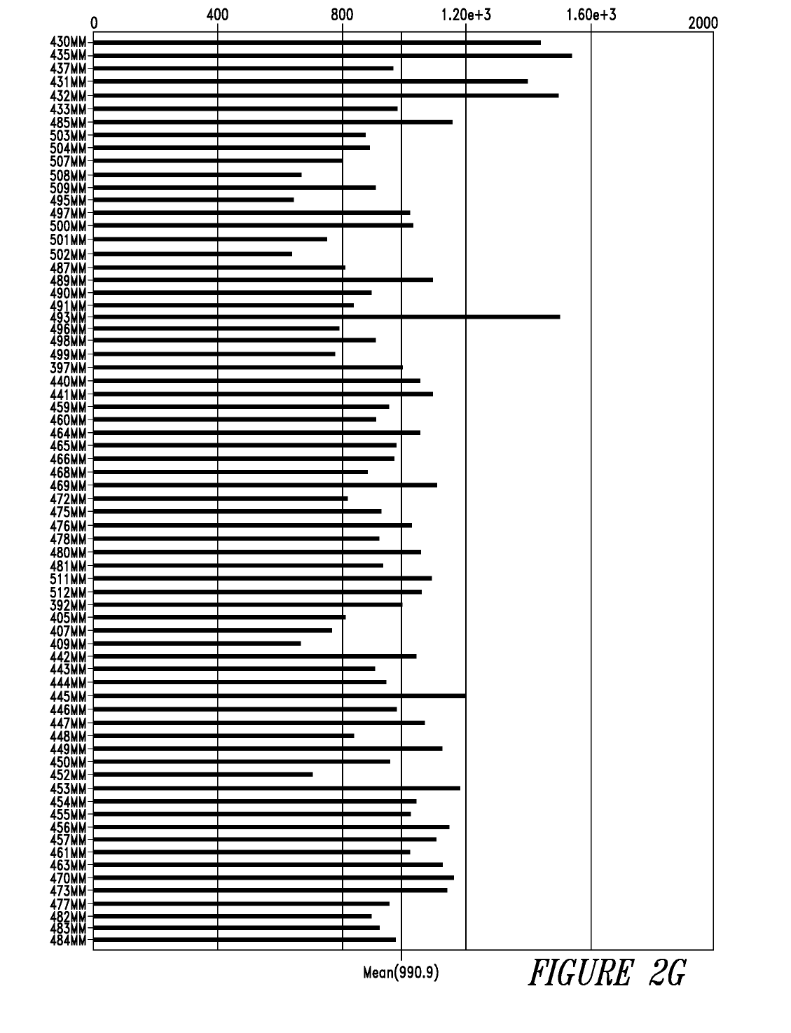

Additional cancer cell lines were also tested for expression of BTN2A1. As shown in Table 2 and FIG. 2, all of these lines showed a strong binding of the antibody. FIG. 2E also shows that BTN2A1 is not expressed to a significant degree by monocytes. FIGS. 2F and G also show data from BioGPS demonstrating that BTN2A1 is not highly expressed on normal tissue, but is expressed at a high level on multiple melanoma cells. Mean value for normal cells is 25.3 and the mean value for melanoma samples: 990.9 based on data from the same arrays and probe sets.

TABLE 2
Cancer cell lines showing staining with anti-BTN2A1 antibody

| Cancer type | Cell line |
|---|---|
| Colon | RKO |
| Colon | CoLo320 |
| Colon | DiFi |
| Colon | V9P |
| Prostate | DU145 |
| Prostate | PC3 |
| Prostate | LNCap |
| Lung | A549 |
| Lung | NCI-H538 |
| Lung | NCI-H2170 |
| Lung | NCI-H520 |

### BTN2A1 Expression on Normal Tissue

Expression of BTN2A1 at the protein level was assessed in normal human tissue cell lines including melanocytes and fibroblasts. No or only weak binding within these tissues could be detected, meaning that BTN2A1 may be a good immunoconjugate target to deliver toxic payloads.

### BTN2A1 Expression on Human Melanoma Tissue

To confirm that BTN2A1 expression was not unique to melanoma cells in culture qPCR was used to assess the expression of BTN2A1 mRNA within primary melanoma tissues. RNA was extracted from 24 metastatic tumor samples and high levels of BTN2A1 expression was detected across all samples. Using Immunohistochemistry (IHC) on a paraffin-embedded melanoma sample and flow-cytometry on single cell suspension of primary metastatic melanoma cells BTN2A1 surface expression was also detected. Immuno-fluorescence with the BTN2A1 antibody (Hu34C1) on cultured melanoma cells confirmed the cell surface membrane-staining.

### Effect of Recombinant BTN2A1 Protein on T-Cells

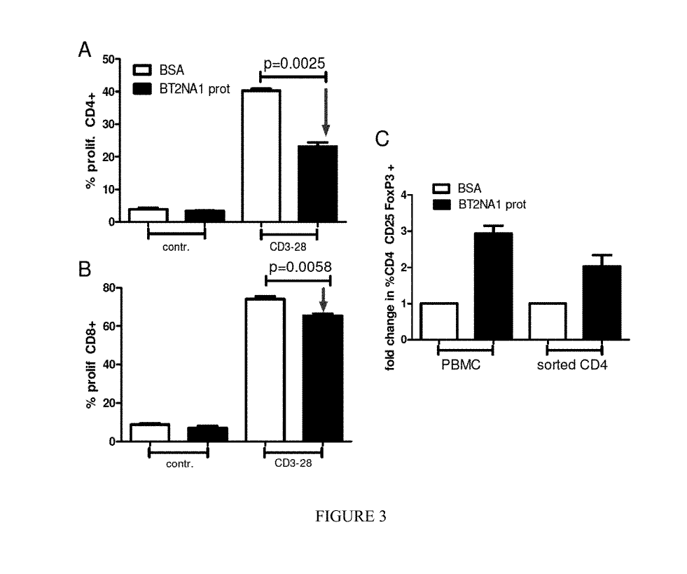

The function of BTN2A1 itself was then assessed to determine if interfering with its expression/activity may influence immunological cellular subsets. Using 96-well plates coated with human recombinant BTN2A1 we tested if the protein itself has an inhibitory or stimulating effect on regulatory T cell development and/or CD4⁺/CD8⁺ T cell proliferation in the presence of a general activation signal (CD3 and CD28 coated beads). As shown in FIG. 3, the presence of the BTN2A1 recombinant protein led to a reduction in proliferation of CD4⁺ and CD8⁺ T cells in stimulated conditions. In comparison, the percentage of CD4/CD25/FoxP3⁺ cells (regulatory T cell phenotype) was shown to be increased.

### Effects of BTN2A1 Blockade on T Cell Function

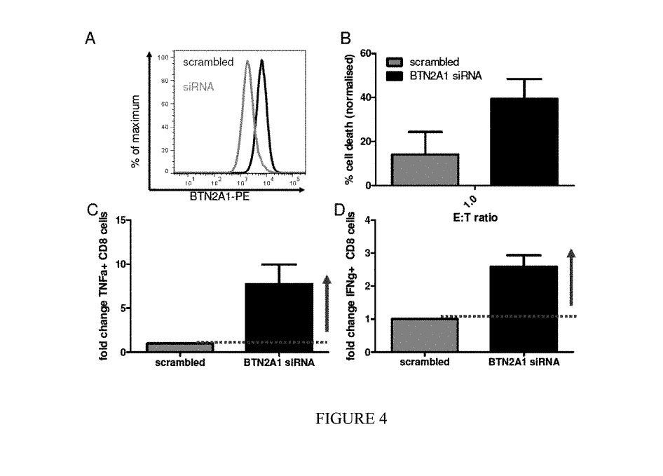

To examine the effect of interfering with BTN2A1 levels, siRNA was used to knockdown BTN2A1 using several melanoma cell lines with a known antigen expression profile and HLA-type. 48 hours after the knockdown, HLA-matched T cell clones specific for either NY-ESO-1 or Melan-A epitopes were added to the melanoma cells for 18-20 h. T cells that recognized their target antigen on tumor cells became activated and marked by the expression of IFNγ and TNFα production. Functionally this activation leads to cytotoxic killing of tumor cells by CD8⁺ T cells (FIG. 4). Despite incomplete knockdown of BTN2A1 (FIG. 4A), melanoma cells were more efficiently killed by the T cells at an effector (E) (T cells) to target (melanoma) (T) cell ratio of 1:1 (FIG. 4B) and this was also reflected by a higher percentage of activated T cells after BTN2A1 knockdown as measured by intracellular cytokine staining (ICS) for IFNγ and TNFα (FIGS. 4C and D).

### An Antibody Against BTN2A1 Induces ADCC

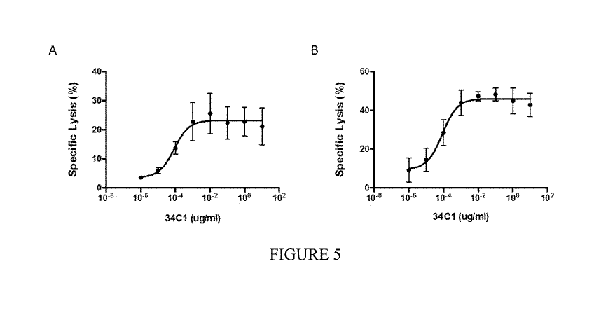

As shown in FIG. 5, anti-BTN2A1 antibody 34C1 induced NK cell-mediated ADCC in melanoma cells and 293 cells expressing BTN2A1. This effect was shown to be dose dependent indicating that it is mediated by the antibody. Similar results were obtained in an experiment performed using NK cells from a single different donor (not shown).

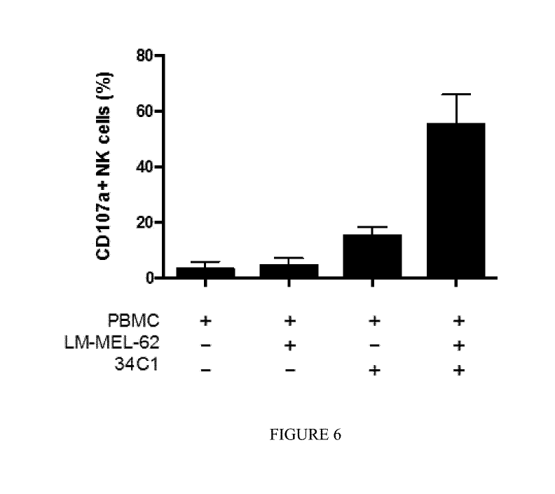

FIG. 6 also shows that the anti-BTN2A1 antibody 34C1 activates NK cells. In particular, these data show that in the presence of BTN2A1 expressing melanoma cells, 34C1 increases the proportion of activated NK cells in a sample of PBMCs.

The foregoing data demonstrate that BTN2A1 is a target for compounds (e.g., antibodies or antigen binding fragments thereof) that induce death of cells for treating, e.g., melanoma.

1-23. (canceled)

24. A computer-implemented method of organising the marketing of a compound that neutralises BTN2A1, the method comprising:
receiving, over a communications network, information associated with the compound;
processing the information to identify a target market for the compound; and
communicating, over the communication network, advertising material to the target market to inform the target market about the compound, wherein the advertising material describes how the compound is used for enhancing or inducing an immune response in a subject when administered to the subject.


SEQUENCE LISTING (4 sequences)

SEQ ID NO: 1 - protein, 527 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 2 - protein, 334 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAELQFFSN

SEQ ID NO: 3 - protein, 466 aa, Homo sapiens
MEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 4 - protein, 330 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAGPV